(19) United States
(12) Patent Application Publication
(10) Pub. No.: US 2026/0265786 A1
(43) Pub. Date: Sep. 10, 2026

(54) COMPOSITIONS AND METHODS USEFUL FOR PRODUCING CYCLOPROPANE COMPOUNDS

(71) Applicant: THE REGENTS OF THE UNIVERSITY OF CALIFORNIA, Oakland, CA (US)

(72) Inventors: Pablo CRUZ-MORALES, Berkeley, CA (US); Kevin YIN, Berkeley, CA (US); Robert BERTRAND, Oakland, CA (US); Ethan OKSEN, Berkeley, CA (US); Aidan COWAN, Berkeley, CA (US); Yuzhong LIU, Berkeley, CA (US); Eric SUNDSTROM, San Mateo, CA (US); Jay D. KEASLING, Berkeley, CA (US)

(21) Appl. No.: 19/681,062

(22) Filed: May 18, 2026

Related U.S. Application Data
(63) Continuation of application No. 17/449,218, filed on Sep. 28, 2021, now Pat. No. 12,680,114.
(60) Provisional application No. 63/084,466, filed on Sep. 28, 2020.

(51) Int. Cl. C12P 5/00; C07K 14/36; C12N 1/16; C12N 1/20; C12N 9/10; C12P 17/16; C12R 1/465; C12R 1/645

(52) U.S. Cl. CPC C12P 5/002; C07K 14/36; C12N 1/16; C12N 1/20; C12N 9/1029; C12P 17/16; C12R 2001/465; C12R 2001/645

(57) ABSTRACT

The present invention also provides for a system or genetically modified host cell capable of producing a cyclopropane compound having the following chemical formula:

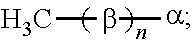

wherein α is —H or —COOR, wherein R is —H or an alkyl group, such as —CH₃, —CH₂CH₃, —(CH₂)₂—CH₃, —(CH₂)₃—CH₃, or —C(CH₃)₃; β is each independently

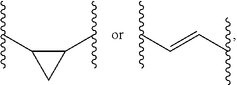

wherein at least one β is

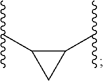 [SMILES: *C1CC1*]

and, n is an integer from 3 to 11, and a method for producing the cyclopropane compound.

### CROSS-REFERENCE TO RELATED APPLICATIONS

The application claims priority as a continuation application to U.S. patent application Ser. No. 17/449,218, filed Sep. 28, 2021, now U.S. Pat. No. ______, issued ______, which claims priority to U.S. Provisional Patent Application Ser. No. 62/084,466, filed Sep. 28, 2020, which is herein incorporated by reference in its entirety.

### STATEMENT OF GOVERNMENTAL SUPPORT

The invention was made with government support under Contract Nos. DE-AC02-05CH11231 awarded by the U.S. Department of Energy. The government has certain rights in the invention.

### REFERENCE TO A “SEQUENCE LISTING” SUBMITTED AS AN ASCII FILE VIA EFS-WEB

The instant application contains a Sequence Listing which has been submitted electronically in XML file format and is hereby incorporated by reference in its entirety. Said XML file copy, created on Oct. 13, 2025, is named “2020-088-02 Sequence Listing.xml” and is 28,672 bytes in size.

### FIELD OF THE INVENTION

The present invention is in the field of the production of cyclopropane compounds, which are useful as a fuel component, such as a jet or rocket fuel.

### BACKGROUND OF THE INVENTION

Kerosene and RP1 are widely used petroleum-derived aviation and rocket fuels. Renewable alternatives for aviation fuel consist of linear and branched chain aliphatic alkanes that are blended with conventional jet fuel to improve their properties. Synthetic polycyclopropanated hydrocarbons have been used as high energy fuels. Their synthesis is complex and involves hazardous chemicals. An example is the chemical synthesis of Syntin (see FIG. 1). Syntin was synthesized in the 1960s and mass produced in the 1970s by the U.S.S.R. and was used in the Soyuz-U2 rockets. No suitable alternative is available for carbon-based rocket fuels.

Instability exists when bonds in a molecule form angles that are abnormal. In cyclopropanes the carbons are sp3 hybridized, changing the ideal bond angle of 109.5° to 60° causing an increase in the potential energy because of the tension on the carbons. The increase in ring strain increases the potential energy (see Table 1).

TABLE 1
Ring strain increases potential energy.

| | | Strained | | | ΔH_{comb/} |
|---|---|---|---|---|---|
| | | bond | | ΔH_{comb} | CH₂ |
| | Molecule | angle | MW | (KJ/Mol) | (KJ/Mol) |
| 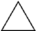 | Cyclopropane | 60° | 42.07 | −499.8 | −166.6 |
| 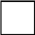 | Cyclobutane | 90° | 56.1 | −655.9 | −164 |
| 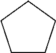 | Cyclopentane | 108° | 70.13 | −793.5 | −158.7 |
| 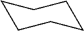 | Cyclohexane | 109.5° | 84.15 | −944.5 | −157.4 |

Combustion energy increases with total C. However, the C number decreases the ring strain angle. Cyclopropane has the highest per C combustion energy. For cyclopropanes, the higher the total C with the most cyclopropanes C equals more combustion energy.

Fuel properties modelling show promising properties for cyclopropanes (see FIG. 2). Saturated 1,2-oligocyclopropanes with five or six cyclopropane groups have diesel fuel-like properties. The predicted energy density, boiling point and derived cetane number (DCN) are found to increase with chain length.

The chemical structures of the cyclopropanes indicated in FIG. 2 are as follows:

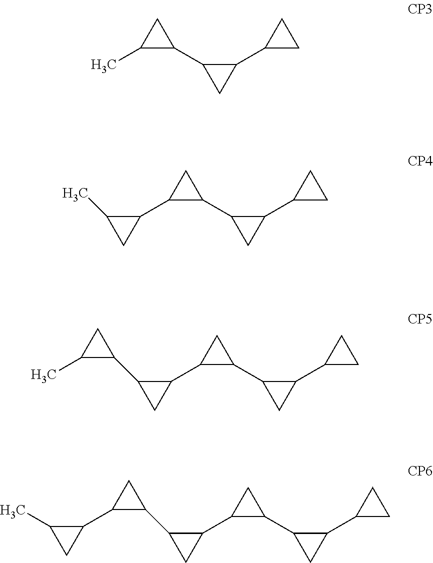

Two naturally occurring small molecules include polycyclopropane functionalities. One is jawsamycin (FR-900848), a filamentous fungi inhibitor, produced by Streptomyces roseoverticillatus (disclosed by Yoshida et al., J. Antibiotics, 43(7): 748-754, 1990). Another is U-106305, a cholesteryl transfer protein inhibitor, produced by Streptomyces sp. U-11136 (disclosed by Kuo et al., J. Am. Chem. Soc., 117(43): 10629-10634, 1995).

### SUMMARY OF THE INVENTION

The present invention provides for a cyclopropane compound having the following chemical formula:

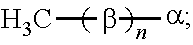

wherein α is —H or —COOR, wherein R is —H or an alkyl group, such as —CH₃, —CH₂CH₃, —(CH₂)₂—CH₃, —(CH₂)₃—CH₃, or —C(CH₃)₃; β is each independently

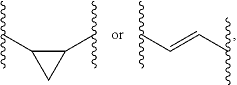

wherein at least one β is

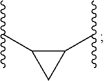 [SMILES: *C1CC1*]

and, n is an integer from 3 to 11; or a mixture thereof. In some embodiments, the mixture comprises two or more, three or more, four or more, or five or more of the cyclopropane compounds of the present invention.

In some embodiments, the cyclopropane compound has a longest carbon chain with 8 to 24 carbon atoms. In some embodiments, the cyclopropane compound has a longest carbon chain with 12 to 24 carbon atoms. In some embodiments, the cyclopropane compound has a longest carbon chain with 14 to 22 carbon atoms. In some embodiments, the cyclopropane compound has a longest carbon chain with 16 to 20 carbon atoms.

In some embodiments, n is 2, 4, 5, 6, 7, 8, 9, 10, or 11, or any range of two integers thereof. In some embodiments, the cyclopropane compound comprises or has 2, 4, 5, 6, 7, 8, 9, 10, or 11 βs which are

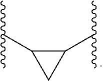 [SMILES: *C1CC1*]

In some embodiments, the cyclopropane compound comprises or has 3 to 10 βs which are

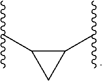 [SMILES: *C1CC1*]

In some embodiments, the cyclopropane compound comprises or has 5 to 7 βs which are

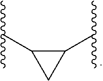 [SMILES: *C1CC1*]

In some embodiments, a

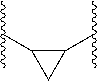 [SMILES: *C1CC1*]

is adjacent to α. In some embodiments, the cyclopropane compound comprises a chain of 2, 3, 4, 5, 6, 7, 8, 9, 10, or 11

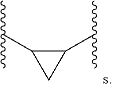 [SMILES: *C1CC1*]

In some embodiments, the cyclopropane compound comprises any of the cyclopropane compounds shown in FIG. 16A to 16I and Table 3, and mixtures thereof.

The present invention provides for a system capable of producing a cyclopropane compound of the present invention comprising: (a) one or more biosynthetic gene clusters (BGCs) each BGC capable of synthesizing a polyketide comprising one or more cyclopropane groups, (b) one or more genes encoding a thioesterase and/or a reductase, and optionally (c) one or more genes encoding an O-methyltransferase, ethyl transferase, a decarboxylase, and/or a decarbonylase; wherein one or more, or all, of the BGC, thioesterase, reductase, O-methyltransferase, ethyl transferase, decarboxylase, and decarbonylase are heterologous to the genetically modified host cell and/or each other.

The present invention provides for a genetically modified host cell capable of producing a cyclopropane compound of the present invention comprising: (a) one or more biosynthetic gene clusters (BGCs) each BGC capable of synthesizing a polyketide comprising one or more cyclopropane groups, (b) one or more genes encoding a thioesterase and/or a reductase, and optionally (c) one or more genes encoding an O-methyltransferase, ethyl transferase, a decarboxylase, and/or a decarbonylase; wherein one or more, or all, of the BGC, thioesterase, reductase, O-methyltransferase, ethyl transferase, decarboxylase, and decarbonylase are heterologous to the genetically modified host cell and/or each other.

In some embodiments, the system or genetically modified host cell comprises: (a) one or more biosynthetic gene clusters (BGCs) each BGC capable of synthesizing a polyketide comprising one or more cyclopropane groups, (b) one or more genes encoding a thioesterase and optionally a reductase, (c) optionally one or more genes encoding an O-methyltransferase, ethyl transferase, and/or a decarboxylase, and (d) optionally one or more genes encoding a decarbonylase. In some embodiments, the system or genetically modified host cell comprises: (a) one or more biosynthetic gene clusters (BGCs) each BGC capable of synthesizing a polyketide comprising one or more cyclopropane groups, (b) one or more genes encoding a thioesterase, and (c) optionally one or more genes encoding an O-methyltransferase, ethyl transferase, and/or a decarboxylase. In some embodiments, the system or genetically modified host cell comprises: (a) one or more biosynthetic gene clusters (BGCs) each BGC capable of synthesizing a polyketide comprising one or more cyclopropane groups, (b) one or more genes encoding a reductase, and (c) optionally one or more genes encoding a decarbonylase. The biosynthetic pathway of various cyclopropane compounds formed by the various enzymes, and an example of an embodiment, is shown in FIG. 5.

The present invention provides for a method of producing a cyclopropane compound comprising: (a) providing a genetically modified host cell of the present invention, (b) culturing or growing the genetically modified host cell in a suitable culture or medium such that the cyclopropane compound, or a mixture thereof, is produced, (c) optionally extracting or separating the cyclopropane compound from the host cells, and/or culture or medium, and (d) optionally introducing a fuel additive to the extracted or separated cyclopropane compound.

In some embodiments, the providing step comprises introducing one or more nucleic acids encoding the BGC, thioesterase, reductase, O-methyltransferase, ethyl transferase, decarboxylase, and/or decarbonylase, each operatively linked to a promoter capable of expressing each enzyme in the genetically modified host cell, into the genetically modified host cell.

In some embodiments, the system or genetically modified host cell comprise a thioesterase, and the BGC and the thioesterase are heterologous to each other.

In some embodiments, the growing or culturing step of the method uses a media comprising a renewable carbon source, such as lignocellulosic biomass. In some embodiments, the cyclopropane compound is produced using a one-pot pretreatment saccharification and fermentation process. In some embodiments, the nucleic acid encoding each enzyme described herein is codon optimized to the genetically modified host cell.

In some embodiments, any one or more, or all, of the enzymes described herein of the system or genetically modified host cell is a homologous enzyme as defined herein. In some embodiments, one or more, or all of the enzymes of all or part of the pathway shown in FIG. 5 is native to the genetically modified host cell. In some embodiments, one or more, or all of the enzymes is heterologous to the genetically modified host cell.

In some embodiments, the culturing or growing step (b) comprises the host cell growing by respiratory cell growth. In some embodiments, the culturing or growing step (b) takes place in a batch process or a fed-batch process, such as a high-gravity fed-batch process. In some embodiments, the culture comprises a biomass, such as a lignocellulosic biomass, or hydrolysate thereof. In some embodiments, the biomass is obtained from softwood feedstock (such as poplar), hardwood feedstock, grass feedstock, and/or agricultural feedstock, or mixture thereof.

In some embodiments, the culture or medium comprises a rich medium, such as LB (Lysogeny-Broth) or comprising one or more ingredients of LB, such as tryptone and/or yeast extract. In some embodiments, the culture or medium comprises hydrolysates derived or obtained from a biomass, such as a lignocellulosic biomass. In some embodiments, the culture or medium comprises one or more carbon sources, such as a sugar, such as glucose or galactose, or glycerol, or a mixture thereof. In some embodiments, the carbon source is fermentable. In some embodiments, the carbon source is non-fermentable. In some embodiments, the culture or medium comprises urea as a nitrogen source. In some embodiments, the culture or medium comprises an ionic liquid (IL).

The present invention provides for a method for constructing a genetically modified host cell of the present invention, comprising (a) introducing a first nucleic acid encoding one or more BGCs each operatively linked to a promoter capable of expressing the BGC gene products in the host cell into host cell, (b) introducing a second nucleic acid encoding one or more thioesterase and/or reductase each operatively linked to a promoter capable of expressing the thioesterase and/or reductase in the host cell into host cell, and (c) optionally introducing a third nucleic acid encoding one or more genes encoding an O-methyltransferase, an ethyl transferase, a decarboxylase, and/or a decarbonylase each operatively linked to a promoter capable of expressing the O-methyltransferase, ethyl transferase, decarboxylase, and/or decarbonylase in the host cell into host cell.

In some embodiments, the invention comprises the use of a heterologous codon-optimized version of each nucleic acid encoding the described enzyme, which are optimized to the genetically modified host cell.

A fuel composition comprising (a) a cyclopropane compound of the present invention, or a mixture thereof, and (b) a fuel additive. In some embodiments, the mixture comprises two or more, three or more, four or more, or five or more of the cyclopropane compounds of the present invention.

The present invention provides for a recombinant nucleic acid encoding the amino acid sequence of Streptomyces sp CC24A Pop1, Pop2, Pop3, and/or Pop4. In some embodiments, the nucleic acid encoding the amino acid sequence of Streptomyces sp CC24A Pop1, Pop2, Pop3, and/or Pop4, is operatively linked to a heterologous promoter capable of expressing the amino acid sequence, or linked to a heterologous vector. In some embodiments, the nucleic acid encodes a hybrid polypeptide of the present invention.

The present invention provides for an isolated or purified polypeptide comprising the amino acid sequence of Streptomyces sp CC24A Pop1, Pop2, Pop3, or Pop4. In some embodiments, the polypeptide comprising the amino acid sequence of Streptomyces sp CC24A Pop1, Pop2, Pop3, or Pop4, is a hybrid polypeptide.

In some embodiments, the BGC capable of synthesizing a polyketide comprising a cyclopropane group comprises an iterative polyketide synthase (PKS; comprising KS-AT-DH-ACP). In some embodiments, the iterative PKS is an iterative PKS of Streptomyces sp. V2, Streptomyces niveiscabiei NRRL B-24457, Streptomyces acidiscabies NCPPB 4445, Streptomyces sp. TLI 146, Actinobacteria bacterium OK074, Streptomyces caatingaensis CMAA 1322, Streptomyces albireticuli SIMD11, Streptomyces albireticuli NRRL B-1670, Streptomyces eurocidicus ATCC 27428, Streptomyces luteoverticillatus CGMCC 15060, Streptomyces sp. 67, Streptomyces sp. 3212, Streptomyces sp. MUSC 14, Streptomyces cellostaticus DSM 40189, Streptomyces roseoverticillatus, Streptomyces klenkii KCTC 29202, Streptomyces yanglinensis CGMCC 4.2023, Streptomyces sp. CC24A, Lentzea jiangxiensis CGMCC 4.6609, or Chloroflexi bacterium HGW-Chloroflexi-6. The amino acid sequence of these BGC are available herein or publicly available. Most of the BGCs are bldA-dependent, which implies that expression occurs during the late stage of growth in natural and heterologous Streptomyces hosts. A predicted phylogenetic relationship of these BGCs are shown in FIG. 6. The pop4 (shown red in FIG. 6) gene product (Pop4) is identified as a trans-acting thioesterase. The pop4 gene encodes an enzyme dedicated to the release of thiol-bound intermediates involved in the biosynthetic route. This is the first example of a thioesterase enzyme releasing a polycyclopropanated product, and not previously been described.

In some embodiments, the naturally occurring polyketide comprising a cyclopropane group is jawsamycin (FR-900848) of Streptomyces roseoverticillatus (disclosed by Yoshida et al., J. Antibiotics, 43(7): 748-754, 1990). The chemical structure of is:

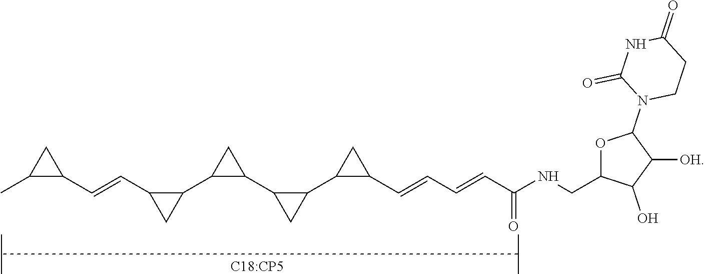

Jawsamycin is biosynthesized with an iterative PKS encoded by BGC001002 (see FIG. 3A), and the biosynthetic pathway is show in FIG. 3B. FIG. 4 shows the enzymatic cyclopropane-functionalized natural products are synthesized via an iterative PKS. The function of each gene product encoded by the BGC is shown in Table 2.

TABLE 2
BGC001002 genes and function of gene products thereof

| Gene | Bp | Annotation |
|---|---|---|
| jaw1 | 1257 | short-chain dehydrogenase - nitroreductase |
| jaw2 | 705 | N-acetyltransferase GCN5 |
| jaw3 | 966 | Regulator (AraC family) |
| jaw4 | 4191 | PKS [KS, DH, AT, ACP] |
| jaw5 | 1437 | SAM dependent methyl transferase |
| jaw6 | 765 | KR |
| jaw7 | 831 | Dioxygenase |
| jaw8 | 1320 | Aminotransferase |
| jaw9 | 729 | Regulator (LysR family) |

In some embodiments, the naturally occurring polyketide comprising a cyclopropane group is U-106305 of Streptomyces sp. U-11136 (disclosed by Kuo et al., J. Am. Chem. Soc., 117(43): 10629-10634, 1995). The chemical structure of is:

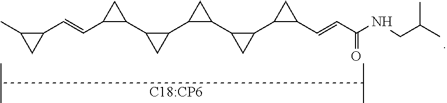

In some embodiments, the

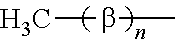

portion of the cyclopropane compound has the following structure:

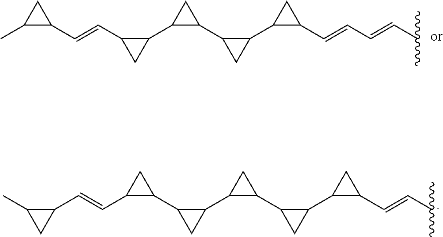 [SMILES: */C=C/C=C/C1CC1C1CC1C1CC1C1CC1/C=C/C1CC1C ; */C=C/C1CC1C1CC1C1CC1C1CC1C1CC1/C=C/C1CC1C]

In some embodiments, the cyclopropane compound is any one listed in Table 3, or a mixture thereof.

TABLE 3
Cyclopropane compounds produced and identified.

| Cyclopropane | | |
|---|---|---|
| Compound | Formula | [M − H]− |
| C24:CP5 | C29H36O2 | 415.264254 |
| C24:CP6 | C30H38O2 | 429.279904 |
| C24:CP7 | C31H40O2 | 443.295554 |
| C24:CP8 | C32H42O2 | 457.311204 |
| C24:CP9 | C33H44O2 | 473.342504 |
| C24:CP10 | C34H46O2 | 485.342504 |
| C24:CP11 | C35H48O2 | 499.358154 |
| C22:CP1 | C23:H26O2 | 333.186004 |
| C22:CP2 | C24H28O2 | 347.201654 |
| C22:CP3 | C25H30O2 | 361.217304 |
| C22:CP4 | C26H32O2 | 375.232954 |
| C22:CP5 | C27H34O2 | 389.248604 |
| C22:CP6 | C28H36O2 | 403.264254 |
| C22:CP7 | C29H38O2 | 417.279904 |
| C22:CP8 | C30H40O2 | 431.295554 |
| C22:CP9 | C31H42O2 | 445.311204 |
| C22:CP10 | C32H44O2 | 459.326854 |
| C20:CP1 | C21H24O2 | 307.170354 |
| C20:CP2 | C22H26O2 | 321.186004 |
| C20:CP3 | C23H28O2 | 335.201654 |
| C20:CP4 | C24H30O2 | 349.217304 |
| C20:CP5 | C25H32O2 | 363.232954 |
| C20:CP6 | C26H34O2 | 377.248604 |
| C20:CP7 | C27H36O2 | 391.264254 |
| C20:CP8 | C28H38O2 | 405.279904 |
| C20:CP9 | C29H40O2 | 419.295554 |
| C18:CP1 | C19H22O2 | 281.154704 |
| C18:CP2 | C20H24O2 | 295.170354 |
| C18:CP3 | C21H26O2 | 309.186004 |
| C18:CP4 | C22H28O2 | 323.201654 |
| C18:CP5 | C23H30O2 | 337.217304 |
| C18:CP6 | C24H32O2 | 351.232954 |
| C18:CP7 | C25H34O2 | 365.248604 |
| C18:CP8 | C26H36O2 | 379.264254 |
| C16:CP1 | C17H20O2 | 255.139053 |
| C16:CP2 | C18H22O2 | 269.154704 |
| C16:CP3 | C19H24O2 | 283.170354 |
| C16:CP4 | C20H26O2 | 297.186004 |
| C16:CP5 | C21H28O2 | 311.201654 |
| C16:CP6 | C22H30O2 | 325.217304 |
| C16:CP7 | C23H32O2 | 339.232954 |
| C14:CP1 | C15H18O2 | 229.123403 |
| C14:CP2 | C16H20O2 | 243.139053 |
| C14:CP3 | C17H22O2 | 257.154704 |
| C14:CP4 | C18H24O2 | 271.170354 |
| C14:CP5 | C19H26O2 | 285.186004 |
| C14:CP6 | C20H28O2 | 299.201654 |
| C12:CP1 | C13H16O2 | 203.107753 |
| C12:CP2 | C14H18O2 | 217.123403 |
| C12:CP3 | C15H20O2 | 231.139053 |
| C12:CP4 | C16H22O2 | 245.154704 |
| C12:CP5 | C17H24O2 | 259.170354 |
| C10:CP1 | C11H14O2 | 177.092103 |
| C10:CP2 | C12H16O2 | 191.107753 |
| C10:CP3 | C13H18O2 | 205.123403 |
| C10:CP4 | C14H20O2 | 219.139053 |
| C8:CP1 | C9H12O2 | 151.076453 |
| C8:CP2 | C10H14O2 | 165.092103 |
| C8:CP3 | C11H16O2 | 179.107753 |

The nomenclature for Cx:CPy is: “x” indicates the number of carbon atoms in the longest carbon chain of the molecule and “y” indicates the number of cyclopropane groups in the molecule.

The incorporation of strained rings that form angles other than the ideal 109.5° in fuel molecules enhances the net heat of combustion. In alkanes, where the carbons are sp3 hybridized, the highest tension possible is achieved in cyclopropanes, which are three-membered carbon rings with 60° angles. One aspect of the invention involves designed cyclopropane containing molecules that can be used as jet or rocket fuels or used as intermediates for development of drugs. To produce them, iterative polyketide synthetases (iPKS) are selected. Thousands of bacterial genomes are mined and a set of iPKS's is identified that are predicted would produce polycyclopropanated molecules. Synthetic biology is used to refactor the pathways and express them in Streptomyces coeliocolor and Escherichia coli host cells. Production of a variety of multicyclopropane containing molecules is achieved. These molecules are useful for the sustainable production of jet or rocket fuels or as precursors for drug development.

Another aspect of the invention involves introducing an artificial genetic system into a bacterial host that then produces the polycyclopropanated molecules. The genes in the system are new: they are bio-prospected, modified and assembled in an artificial cassette that forms an operon containing 4 genes. Each gene is driven by an artificial promoter for constitutive expression.

The genes cassette is cloned into an integrative plasmid system which is introduced into a host cell, such S. coelicolor, by inter-species conjugation. A phiC31 integrase then drives insertion of the gene cassette into a discrete site in the chromosome of the host. An apramycin selection marker is used to select for clones with successful cassette integration. The host strain carrying the artificial cassette is then used as seed for fermentation leading to the production of the cyclopropanated molecules.

To reduce this system to practice, the properties of strained carbon ring molecules is predicted, a potential biosynthetic route and bio prospect potential genes to construct the biosynthetic pathway is identified from thousands of genomes. To construct the genetic system genetic material from bacteria is cloned, mutated, and synthesized. To test the production of the molecules a series of experiments is performed to evaluate the impact of codon usage in the production of the molecule. Multiple host bacteria, promoters, culture media, and extraction media are tested to ensure optimal production. Analytical methods and isotope labeling experimental procedures are developed to confirm the production of the molecules.

This invention is useful to produce high energy fuels for aviation, rocketry, automotive, maritime, and other applications where high energy density is valuable. This invention is also useful to produce polycyclopropanated molecules for drug development.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1. Chemical synthesis of Syntin.

FIG. 2. Fuel properties modeling shows promising properties for polycyclopropanes.

FIG. 3A. Organization of the Streptomyces roseoverticillatus BGC0001002 operon.

FIG. 3B. Biosynthetic pathway for the synthesis of jawsamycin.

FIG. 4. Cyclopropane-functionalized natural products are synthesized via an iterative PKS.

FIG. 5. Engineered systems for producing high energy cyclopropanes fuels from cyclopropane containing polyketides.

FIG. 6. A predicted phylogenetic relationship of BGCs identified for producing polycyclopropane containing polyketides].

FIG. 7. In vitro pathway reconstruction using cell lysates E. coli BL21 yields products consistent with polycyclopropane acids. Cell lysates from BL21 expressing SUMO-tagged Pop1, Pop3, Pop4, His-tag purified phosphopantetheine transferase (Sfp). 1.0 mM acetyl-CoA, 2.0 mM malonyl-CoA, 2.0 mM SAM, 3.0 mM NADPH, 2.0 mM DTT phosphate buffer (100 mM), pH 7.5, incubated at 30° C. for 16 hours.

FIG. 8. In vivo production of polycyclopropane acids is achieved in Streptomyces coelicolor M1152 (high resolution LC-MS). POP3.3: pathway from Streptomyces sp. C24A; POP3.1: pathway from Streptomyces albireticuli with TTA codons; POP3.1+bldA: pathway from Streptomyces albireticuli plus constitutive bldA; POP3.2: pathway from Streptomyces albireticuli with TTA codons mutated. The calculated production is about 1.2 mg per liter.

FIG. 9. C¹³-labelled products are consistent with structural polycyclopropane acid products. Cyclopropane acids include a S-adenosyl-methionine (SAM) derived carbon atom incorporated in a predictable position. Products obtained from methionine (methyl-C¹³) fed fermentation are analyzed using high resolution LC-MS. They presented the predicted isotopes.

FIG. 10. Extracted ion chromatograms, unlabeled, M/z (—H)=351.23 and isotopes.

FIG. 11. Extracted ion chromatograms, C¹³-labeled, Mz (—H)=351.23 and isotopes.

FIG. 12. C¹³-labeled, high resolution mass spectra, retention time is 19.2 minutes.

FIG. 13. Heterologous systems produce a blend of products. High resolution LC-MS.

FIG. 14. Pathway has preference for C18-C22 products. No changes are observed in product profile after altering TE expression, and total production appears improved.

FIG. 15. Media supplementation increases total biomass in producer strain POP3.2. Total dry biomass (n=2).

FIG. 16A. C8 cyclopropane compounds. Further C8 cyclopropane compounds can include any cyclopropane compound wherein one or more C══C double bonds are replaced with a cyclopropane group. Further C8 cyclopropane compounds can include any cyclopropane compound wherein the —COOH is replace with —H, —COO—CH₃, or —COO—CH₂CH₃.

FIG. 16B. C10 cyclopropane compounds. Further C10 cyclopropane compounds can include any cyclopropane compound wherein one or more C══C double bonds are replaced with a cyclopropane group. Further C10 cyclopropane compounds can include any cyclopropane compound wherein the —COOH is replace with —H, —COO—CH₃, or —COO—CH₂CH₃.

FIG. 16C. C12 cyclopropane compounds. Further C12 cyclopropane compounds can include any cyclopropane compound wherein one or more C══C double bonds are replaced with a cyclopropane group. Further C12 cyclopropane compounds can include any cyclopropane compound wherein the —COOH is replace with —H, —COO—CH₃, or —COO—CH₂CH₃.

FIG. 16D. C14 cyclopropane compounds. Further C14 cyclopropane compounds can include any cyclopropane compound wherein one or more C══C double bonds are replaced with a cyclopropane group. Further C14 cyclopropane compounds can include any cyclopropane compound wherein the —COOH is replace with —H, —COO—CH₃, or —COO—CH₂CH₃.

FIG. 16E. C16 cyclopropane compounds. Further C16 cyclopropane compounds can include any cyclopropane compound wherein one or more C══C double bonds are replaced with a cyclopropane group. Further C16 cyclopropane compounds can include any cyclopropane compound wherein the —COOH is replace with —H, —COO—CH₃, or —COO—CH₂CH₃.

FIG. 16F. C18 cyclopropane compounds. Further C18 cyclopropane compounds can include any cyclopropane compound wherein one or more C══C double bonds are replaced with a cyclopropane group. Further C18 cyclopropane compounds can include any cyclopropane compound wherein the —COOH is replace with —H, —COO—CH₃, or —COO—CH₂CH₃.

FIG. 16G. C20 cyclopropane compounds. Further C20 cyclopropane compounds can include any cyclopropane compound wherein one or more C══C double bonds are replaced with a cyclopropane group. Further C20 cyclopropane compounds can include any cyclopropane compound wherein the —COOH is replace with —H, —COO—CH₃, or —COO—CH₂CH₃.

FIG. 16H. C22 cyclopropane compounds. Further C22 cyclopropane compounds can include any cyclopropane compound wherein one or more C══C double bonds are replaced with a cyclopropane group. Further C22 cyclopropane compounds can include any cyclopropane compound wherein the —COOH is replace with —H, —COO—CH₃, or —COO—CH₂CH₃.

FIG. 16I. C24 cyclopropane compounds. Further C24 cyclopropane compounds can include any cyclopropane compound wherein one or more C══C double bonds are replaced with a cyclopropane group. Further C24 cyclopropane compounds can include any cyclopropane compound wherein the —COOH is replace with —H, —COO—CH₃, or —COO—CH₂CH₃.

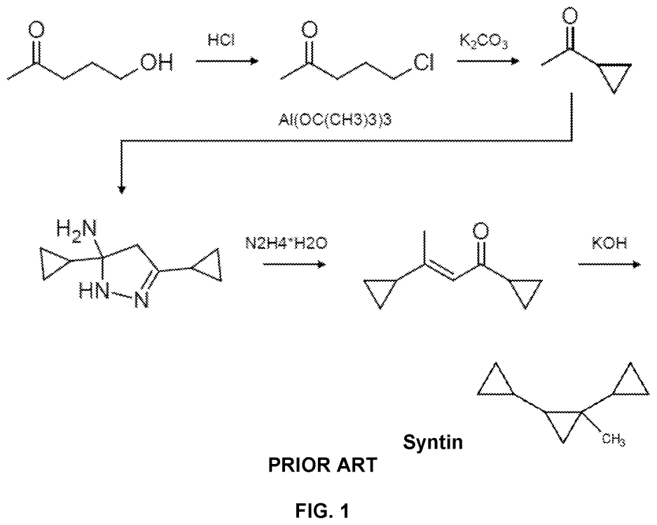

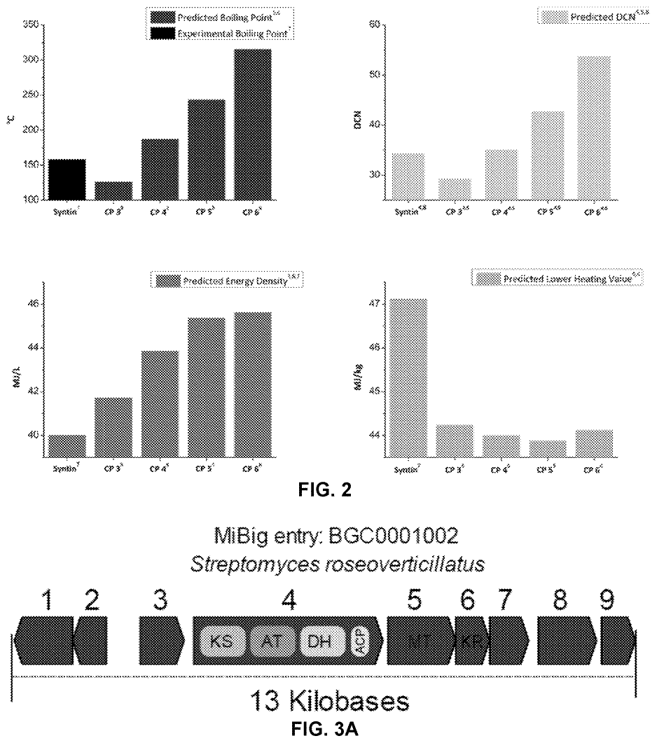

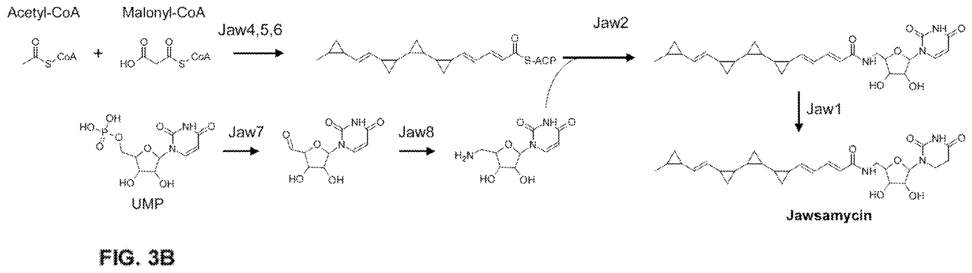

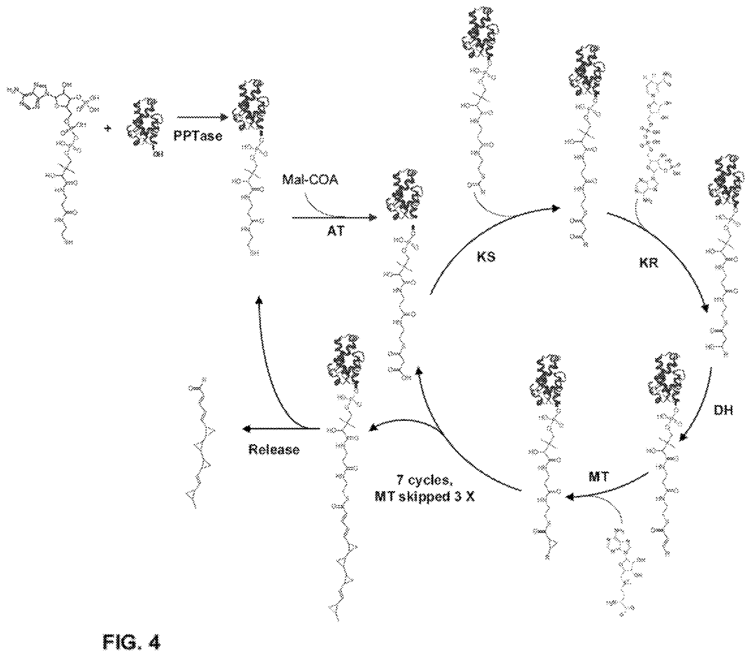

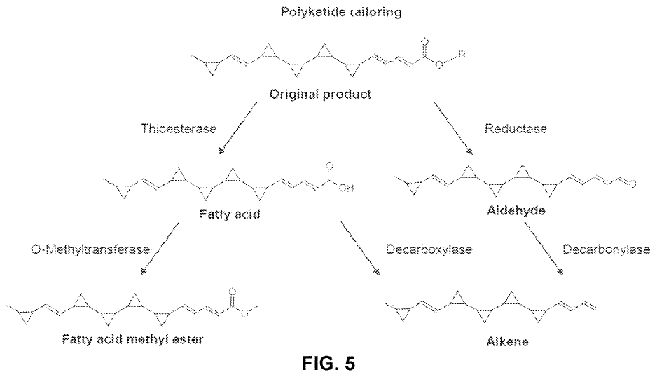

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only, and is not intended to be limiting.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

As used in the specification and the appended claims, the singular forms “a,” “an,” and “the” include plural referents unless the context clearly dictates otherwise. Thus, for example, reference to an “expression vector” includes a single expression vector as well as a plurality of expression vectors, either the same (e.g., the same operon) or different; reference to “cell” includes a single cell as well as a plurality of cells; and the like.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

The term “about” refers to a value including 10% more than the stated value and 10% less than the stated value.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

The terms “host cell” and “host microorganism” are used interchangeably herein to refer to a living biological cell, such as a microbe, that can be transformed via insertion of an expression vector. Thus, a host organism or cell as described herein may be a prokaryotic organism (e.g., an organism of the kingdom Eubacteria) or a eukaryotic cell. As will be appreciated by one of ordinary skill in the art, a prokaryotic cell lacks a membrane-bound nucleus, while a eukaryotic cell has a membrane-bound nucleus.

The term “heterologous” as used herein refers to a material, or nucleotide or amino acid sequence, that is found in or is linked to another material, or nucleotide or amino acid sequence, wherein the materials, or nucleotide or amino acid sequences, are foreign to each other (i.e., not found or linked together in nature).

The terms “expression vector” or “vector” refer to a compound and/or composition that transduces, transforms, or infects a host cell, thereby causing the cell to express nucleic acids and/or proteins other than those native to the cell, or in a manner not native to the cell. An “expression vector” contains a sequence of nucleic acids (ordinarily RNA or DNA) to be expressed by the host cell. Optionally, the expression vector also comprises materials to aid in achieving entry of the nucleic acid into the host cell, such as a virus, liposome, protein coating, or the like. The expression vectors contemplated for use in the present invention include those into which a nucleic acid sequence can be inserted, along with any preferred or required operational elements. Further, the expression vector must be one that can be transferred into a host cell and replicated therein. Particular expression vectors are plasmids, particularly those with restriction sites that have been well documented and that contain the operational elements preferred or required for transcription of the nucleic acid sequence. Such plasmids, as well as other expression vectors, are well known to those of ordinary skill in the art.

The terms “polynucleotide” and “nucleic acid” are used interchangeably and refer to a single or double-stranded polymer of deoxyribonucleotide or ribonucleotide bases read from the 5′ to the 3′ end. A nucleic acid of the present invention will generally contain phosphodiester bonds, although in some cases, nucleic acid analogs may be used that may have alternate backbones, comprising, e.g., phosphoramidate, phosphorothioate, phosphorodithioate, or O-methylphophoroamidite linkages (see Eckstein, Oligonucleotides and Analogues: A Practical Approach, Oxford University Press); positive backbones; non-ionic backbones, and non-ribose backbones. Thus, nucleic acids or polynucleotides may also include modified nucleotides that permit correct read-through by a polymerase. “Polynucleotide sequence” or “nucleic acid sequence” includes both the sense and antisense strands of a nucleic acid as either individual single strands or in a duplex. As will be appreciated by those in the art, the depiction of a single strand also defines the sequence of the complementary strand; thus the sequences described herein also provide the complement of the sequence. Unless otherwise indicated, a particular nucleic acid sequence also implicitly encompasses variants thereof (e.g., degenerate codon substitutions) and complementary sequences, as well as the sequence explicitly indicated. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc.

The term “promoter,” as used herein, refers to a polynucleotide sequence capable of driving transcription of a DNA sequence in a cell. Thus, promoters used in the polynucleotide constructs of the invention include cis- and trans-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) gene transcription. Promoters are located 5′ to the transcribed gene, and as used herein, include the sequence 5′ from the translation start codon (i.e., including the 5′ untranslated region of the mRNA, typically comprising 100-200 bp). Most often the core promoter sequences lie within 1-2 kb of the translation start site, more often within 1 kbp and often within 500 bp of the translation start site. By convention, the promoter sequence is usually provided as the sequence on the coding strand of the gene it controls. In the context of this application, a promoter is typically referred to by the name of the gene for which it naturally regulates expression. A promoter used in an expression construct of the invention is referred to by the name of the gene. Reference to a promoter by name includes a wildtype, native promoter as well as variants of the promoter that retain the ability to induce expression. Reference to a promoter by name is not restricted to a particular species, but also encompasses a promoter from a corresponding gene in other species.

A polynucleotide is “heterologous” to a host cell or a second polynucleotide sequence if it originates from a foreign species, or, if from the same species, is modified from its original form. For example, when a polynucleotide encoding a polypeptide sequence is said to be operably linked to a heterologous promoter, it means that the polynucleotide coding sequence encoding the polypeptide is derived from one species whereas the promoter sequence is derived from another, different species; or, if both are derived from the same species, the coding sequence is not naturally associated with the promoter (e.g., is a genetically engineered coding sequence, e.g., from a different gene in the same species, or an allele from a different ecotype or variety).

The term “operatively linked” refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a DNA or RNA sequence if it stimulates or modulates the transcription of the DNA or RNA sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

### Enzymes, and Nucleic Acids Encoding Thereof

A homologous enzyme is an enzyme that has a polypeptide sequence that is at least 70%, 75%, 80%, 85%, 90%, 95% or 99% identical to any one of the enzymes described in this specification or in an incorporated reference. The homologous enzyme comprises or retains amino acid residues that are recognized as conserved for the enzyme. The homologous enzyme may have non-conserved amino acid residues replaced or found to be of a different amino acid, or amino acid(s) inserted or deleted, but which does not affect or has insignificant effect on the enzymatic activity of the homologous enzyme. The homologous enzyme has an enzymatic activity that is identical or essentially identical to the enzymatic activity any one of the enzymes described in this specification or in an incorporated reference. The homologous enzyme may be found in nature or be an engineered mutant thereof.

The nucleic acid constructs of the present invention comprise nucleic acid sequences encoding one or more of the subject enzymes. The nucleic acid of the subject enzymes are operably linked to promoters and optionally control sequences such that the subject enzymes are expressed in a host cell cultured under suitable conditions. The promoters and control sequences are specific for each host cell species. In some embodiments, expression vectors comprise the nucleic acid constructs. Methods for designing and making nucleic acid constructs and expression vectors are well known to those skilled in the art.

Sequences of nucleic acids encoding the subject enzymes are prepared by any suitable method known to those of ordinary skill in the art, including, for example, direct chemical synthesis or cloning. For direct chemical synthesis, formation of a polymer of nucleic acids typically involves sequential addition of 3′-blocked and 5′-blocked nucleotide monomers to the terminal 5′-hydroxyl group of a growing nucleotide chain, wherein each addition is effected by nucleophilic attack of the terminal 5′-hydroxyl group of the growing chain on the 3′-position of the added monomer, which is typically a phosphorus derivative, such as a phosphotriester, phosphoramidite, or the like. Such methodology is known to those of ordinary skill in the art and is described in the pertinent texts and literature (e.g., in Matteuci et al. (1980) Tet. Lett. 521:719; U.S. Pat. Nos. 4,500,707; 5,436,327; and 5,700,637). In addition, the desired sequences may be isolated from natural sources by splitting DNA using appropriate restriction enzymes, separating the fragments using gel electrophoresis, and thereafter, recovering the desired nucleic acid sequence from the gel via techniques known to those of ordinary skill in the art, such as utilization of polymerase chain reactions (PCR; e.g., U.S. Pat. No. 4,683,195).

Each nucleic acid sequence encoding the desired subject enzyme can be incorporated into an expression vector. Incorporation of the individual nucleic acid sequences may be accomplished through known methods that include, for example, the use of restriction enzymes (such as BamHI, EcoRI, HhaI, Xhol, XmaI, and so forth) to cleave specific sites in the expression vector, e.g., plasmid. The restriction enzyme produces single stranded ends that may be annealed to a nucleic acid sequence having, or synthesized to have, a terminus with a sequence complementary to the ends of the cleaved expression vector. Annealing is performed using an appropriate enzyme, e.g., DNA ligase. As will be appreciated by those of ordinary skill in the art, both the expression vector and the desired nucleic acid sequence are often cleaved with the same restriction enzyme, thereby assuring that the ends of the expression vector and the ends of the nucleic acid sequence are complementary to each other. In addition, DNA linkers may be used to facilitate linking of nucleic acids sequences into an expression vector.

A series of individual nucleic acid sequences can also be combined by utilizing methods that are known to those having ordinary skill in the art (e.g., U.S. Pat. No. 4,683,195).

For example, each of the desired nucleic acid sequences can be initially generated in a separate PCR. Thereafter, specific primers are designed such that the ends of the PCR products contain complementary sequences. When the PCR products are mixed, denatured, and reannealed, the strands having the matching sequences at their 3′ ends overlap and can act as primers for each other Extension of this overlap by DNA polymerase produces a molecule in which the original sequences are “spliced” together. In this way, a series of individual nucleic acid sequences may be “spliced” together and subsequently transduced into a host microorganism simultaneously. Thus, expression of each of the plurality of nucleic acid sequences is effected.

Individual nucleic acid sequences, or “spliced” nucleic acid sequences, are then incorporated into an expression vector. The invention is not limited with respect to the process by which the nucleic acid sequence is incorporated into the expression vector. Those of ordinary skill in the art are familiar with the necessary steps for incorporating a nucleic acid sequence into an expression vector. A typical expression vector contains the desired nucleic acid sequence preceded by one or more regulatory regions, along with a ribosome binding site, e.g., a nucleotide sequence that is 3-9 nucleotides in length and located 3-11 nucleotides upstream of the initiation codon in E. coli. See Shine et al. (1975) Nature 254:34 and Steitz, in Biological Regulation and Development: Gene Expression (ed. R. F. Goldberger), vol. 1, p. 349, 1979, Plenum Publishing, N.Y.

Regulatory regions include, for example, those regions that contain a promoter and an operator. A promoter is operably linked to the desired nucleic acid sequence, thereby initiating transcription of the nucleic acid sequence via an RNA polymerase enzyme. An operator is a sequence of nucleic acids adjacent to the promoter, which contains a protein-binding domain where a repressor protein can bind. In the absence of a repressor protein, transcription initiates through the promoter. When present, the repressor protein specific to the protein-binding domain of the operator binds to the operator, thereby inhibiting transcription. In this way, control of transcription is accomplished, based upon the particular regulatory regions used and the presence or absence of the corresponding repressor protein. An example includes lactose promoters (LacI repressor protein changes conformation when contacted with lactose, thereby preventing the LacI repressor protein from binding to the operator). Another example is the tac promoter. (See deBoer et al. (1983) Proc. Natl. Acad. Sci. USA, 80:21-25.) As will be appreciated by those of ordinary skill in the art, these and other expression vectors may be used in the present invention, and the invention is not limited in this respect.

Although any suitable expression vector may be used to incorporate the desired sequences, readily available expression vectors include, without limitation: plasmids, such as pSC101, pBR322, pBBR1MCS-3, pUR, pEX, pMR100, pCR4, pBAD24, pUC19; bacteriophages, such as M13 phage and X phage. Of course, such expression vectors may only be suitable for particular host cells. One of ordinary skill in the art, however, can readily determine through routine experimentation whether any particular expression vector is suited for any given host cell. For example, the expression vector can be introduced into the host cell, which is then monitored for viability and expression of the sequences contained in the vector. In addition, reference may be made to the relevant texts and literature, which describe expression vectors and their suitability to any particular host cell.

The expression vectors of the invention must be introduced or transferred into the host cell. Such methods for transferring the expression vectors into host cells are well known to those of ordinary skill in the art. For example, one method for transforming E. coli with an expression vector involves a calcium chloride treatment wherein the expression vector is introduced via a calcium precipitate. Other salts, e.g., calcium phosphate, may also be used following a similar procedure. In addition, electroporation (i.e., the application of current to increase the permeability of cells to nucleic acid sequences) may be used to transfect the host microorganism. Also, microinjection of the nucleic acid sequencers) provides the ability to transfect host microorganisms. Other means, such as lipid complexes, liposomes, and dendrimers, may also be employed. Those of ordinary skill in the art can transfect a host cell with a desired sequence using these or other methods.

For identifying a transfected host cell, a variety of methods are available. For example, a culture of potentially transfected host cells may be separated, using a suitable dilution, into individual cells and thereafter individually grown and tested for expression of the desired nucleic acid sequence. In addition, when plasmids are used, an often-used practice involves the selection of cells based upon antimicrobial resistance that has been conferred by genes intentionally contained within the expression vector, such as the amp, gpt, neo, and hyg genes.

When the host cell is transformed with at least one expression vector. When only a single expression vector is used (without the addition of an intermediate), the vector will contain all of the nucleic acid sequences necessary.

Once the host cell has been transformed with the expression vector, the host cell is allowed to grow. For microbial hosts, this process entails culturing the cells in a suitable medium. It is important that the culture medium contain an excess carbon source, such as a sugar (e.g., glucose) when an intermediate is not introduced. In this way, cellular production of the cyclopropane compound ensured. When added, any intermediate is present in an excess amount in the culture medium.

Any means for extracting or separating the modified cyclopropane compound from the host cell may be used. For example, the host cell may be harvested and subjected to hypotonic conditions, thereby lysing the cells. The lysate may then be centrifuged and the supernatant subjected to high performance liquid chromatography (HPLC) or gas chromatography (GC).

### Host Cells

In some embodiments, the host cells are genetically modified in that heterologous nucleic acid have been introduced into the host cells, and as such the genetically modified host cells do not occur in nature. The suitable host cell is one capable of expressing a nucleic acid construct encoding one or more enzymes described herein. The gene(s) encoding the enzyme(s) may be heterologous to the host cell or the gene may be native to the host cell but is operatively linked to a heterologous promoter and one or more control regions which result in a higher expression of the gene in the host cell.

Each introduced enzyme can be native or heterologous to the host cell. Where the enzyme is native to the host cell, the host cell is genetically modified to modulate expression of the enzyme. This modification can involve the modification of the chromosomal gene encoding the enzyme in the host cell or a nucleic acid construct encoding the gene of the enzyme is introduced into the host cell. One of the effects of the modification is the expression of the enzyme is modulated in the host cell, such as the increased expression of the enzyme in the host cell as compared to the expression of the enzyme in an unmodified host cell.

The genetically modified host cell can be any bacterial cell capable of production of the cyclopropane compound of the present invention in accordance with the methods of the invention.

In some embodiments, the host cell is a prokaryotic cell, such as a bacterial cell.

In some embodiments, the host cell is a bacterial cell selected from the Escherichia, Enterobacter, Azotobacter, Erwinia, Bacillus, Pseudomonas, Klebsielia, Proteus, Salmonella, Serratia, Shigella, Ralstonia, Rhizobia, or Vitreoscilla taxonomical class. Bacterial host cells suitable for the invention include, but are not limited to, Escherichia, Corynebacterium, Pseudomonas, Streptomyces, and Bacillus. In some embodiments, the Escherichia cell is an E. coli, E. albertii, E. fergusonii, E. hermanii, E. marmotae, or E. vulneris. In some embodiments, the Corynebacterium cell is Corynebacterium glutamicum, Corynebacterium kroppenstedtii, Corynebacterium alimapuense, Corynebacterium amycolatum, Corynebacterium diphtheriae, Corynebacterium efficiens, Corynebacterium jeikeium, Corynebacterium macginleyi, Corynebacterium matruchotii, Corynebacterium minutissimum, Corynebacterium renale, Corynebacterium striatum, Corynebacterium ulcerans, Corynebacterium urealyticum, or Corynebacterium uropygiale. In some embodiments, the Pseudomonas cell is a P. putida, P. aeruginosa, P. chlororaphis, P. fluorescens, P. pertucinogena, P. stutzeri, P. syringae, P. cremoricolorata, P. entomophila, P. fulva, P. monteilii, P. mosselii, P. oryzihabitans, P. parafluva, or P. plecoglossicida. In some embodiments, the Streptomyces cell is a S. coelicolor, S. lividans, S. venezuelae, S. ambofaciens, S. avermitilis, S. albus, or S. scabies. In some embodiments, the Bacillus cell is a B. subtilis, B. megaterium, B. lichenformis, B. anthracis, B. amyloliquefaciens, B. pumilus, B. brevis, B. aminovorans, or B. fusiformis. In some embodiments the bacterial cell is a Gram-positive bacterium, such as a Streptomyces species, such as any Streptomyces species or strain taught herein.

The genetically modified host cell can be any yeast capable of production of the cyclopropane compound in accordance with the methods of the invention.

In some embodiments, the host cell is a yeast. Yeast host cells suitable for the invention include, but are not limited to, Yarrowia, Candida, Bebaromyces, Saccharomyces, Schizosaccharomyces and Pichia cells. In one embodiment, Saccharomyces cerevisae is the host cell. In one embodiment, the yeast host cell is a species of Candida, including but not limited to C. tropicalis, C. maltosa, C. apicola, C. paratropicalis, C. albicans, C. cloacae, C. guillermondii, C. intermedia, C. lipolytica, C. panapsilosis and C. zeylenoides. In one embodiment, Candida tropicalis is the host cell.

In some embodiments, the yeast host cell is a non-oleaginous yeast. In some embodiments, the yeast host cell is a basidiomycete. In some embodiments, the yeast host cell is an oleaginous yeast. In some embodiments, the oleaginous yeast is a Rhodosporidium species. In some embodiments, the Rhodosporidium species is Rhodosporidium toruloides. In some embodiments, the Rhodosporidium toruloides is strain IFO 0880.

### Amino Acid Sequence of Exemplary Enzymes

The amino acid sequence of Streptomyces sp. 24A Pop1 is as follows:

(SEQ ID NO: 1)
VDRHVSIVGIGCALPGGVADVEDVRGAFLHGRDCVGPIPEERWGADAFYD
PDPLRPGRTYVRHGGFVDDIDAFDAAFFGISDTEAARMDPQQRLLLQTVW
HALENAGQNPDELRGTSTGVFLASMNTNNYAMLKNTLQGPEGITPYDAMG
DAISISAGRVAHFLGVEGPCLAVDTACSGALVALHLARQSILAGECDAAI
VAGVNVMLNPGIHIAFSKVGLLSRNGQCRAFDARADGYVRSEGCVAALLR
RESLAVERGDPIYATVVGTAVNHDGRTQALTAPNGRNQEQVIHRALAGVG
IDTADTGYVEAHGTGTPVGDPIEMSAIVNAYGYGRPADRPLYVGSAKSNF
GHIEAGAGLLGVVRAALSLQHEVIFPSIHVDQLNPRIDLRGAPVRLPSAP
VPWPRGDTPRHAGVNSFGYSGTNAHAILREAPAVRPREQAAPRRATELLA
LSAKSAESLEELADRWAEFLAHDDIDLGAAARTAAIGRATLRHRLTVTAA
DSAEAAKALRRRRSGRAPVTVSEGRARRNPRVAFVFTGQGAQYPGMGREL
YAAEPVFAAALDRCADVMDADLGLPLHEVLFDERVSTEALNNTQYVQPAV
FAVEFALAELLKDWGVEPSVVIGHSIGELVAACTAGMLPFEEAARFAVRR
GRAMGSLPPGGKMLAVAAEAAVVEAWLTGREDAVSLAAVNGPRSVVVSGA
AAAVDEVARRADEAGLRTTELKVSHAFHSPLMDPALAELERAAAALHPRV
PAVPVVSNVTGAPLTGTEEPAYWAAQMRDPVRFHDGMRTVVESGCSAVVE
VGPHAALIPAVAAAFGQADIALVRTLLRDRQDVRNMRTAAGVLHTTGCPV
RLPRLFTTGGHRRIHAPEYPFRRDRHWIAPTEGGWDLGSMMRGPRDETGP
SGTWPTELAAATPWADHRVLGATVFPATGHLELALRALAAHTADAPDAPL
TGTAAPATPASCEDLAFVRPLLLKPRRPTVATTALRPAPGQDGAFHFTVS
TSGTGQHPVEHCRGTVRPAAAPADEPRTPPAELRAPLGAGQPPGRLYGML
REAGLEYGTSFSTVRELWPGGDGTGAALGRIRATPDGAGGAEHGHALATM
LDGCLHVTAAALFTLPARLLEGAYIPVTLRRATLHRPLPEQVWSQVSVRT
NDQGTAAVASARVVDDEGRLLAELDGLELRHTSALTGAADSGQAPAPARP
YSGEARKLLLERLGPLGQRERVAAMGAWLLDEVRDTLGQAADDFDIDDLD
PSTALLEIGLDSLMITELQRRLQEKLDFRFEAMEALTYQSLEDLAGYILD
RALGPALPAAARNETEQQPEPA

The amino acid sequence of Streptomyces sp. 24A Pop2 is as follows:

(SEQ ID NO: 2)
MLDAGFVHTYIDTHLEQRQVNKIQHGFPSPRYWSRTDVPVEEIAEDRRR
IRAAGRDSFVNFYVGVPYCIQTDPGKCGYCLFPVEEFQGNAALENYFGY
VEREADLYRQALSGATLGAVYFGGGTSNLYKEPMYHRLMDLVRGLFPDI
APGADITLEGIPQLFSRAKMQAIKDSGMNRVSMGIQQVDERLNKLSGRK
QTTRHVVQSLEWARELGLAANVDLIFGWPQQTVGTMLKDLETLVSWNVY
DITHYELNVGGPTDFALNRFHELPSTLANLEMYRASRDFLTDQGYEQIT
AYNFRKPGDPAGRGYEEGVNRFLDSMDTVGLGYAAVSFFGNSAIGTDRS
WSFINHLSLPRYKQALEEGRFPVERGFSHEAADWRLAMLFRSLFGLTVN
RADYRAAFGTDVYEEFATVWDGLGEYGFVEVSDAEVRLVGDGPFYTPMV
QALLAEERYRALRERETQAAQARRAARRARRTGNGQDGAGTEDDAAPAD
TGAAADAEAAAASPARG

The amino acid sequence of Streptomyces sp. 24A Pop3 is as follows:

(SEQ ID NO: 3)
MSEHLPLDGKRLVVTGGARGIGAGIVRLALRQGAEVVFSYNRSAERARE
LCAELRAAHPGQQCTALPAQVADTDSAARFAQAALEALGSVDALVNNAG
VTRDGVFARMRREDWDEAVETNLGSMFTVTRPLVMALVRRRAGAIVNVT
SSVGIHGAPGQANYAASKAGIIGFSKALAKELAELGVRVNAVAPGLIAT
DMTAGIPPERLEEIKKRIPGRQLGSVEDVAHLVCFLASDRARYITGQVI
EVSGGLAH
>pop4_Streptomyces_sp_24A
(SEQ ID NO: 4)
VTTDHSTAVEDLLDVLTPDRIDARTFRPRGPVSWSSRHLYGGQVAAQAL
LAAGRTVAEDRPVHSLHAYFVRPGDPDAAVPLLFDVDEVRDGRAVSLRQ
VTARQRDAVLFTLSASFHRAEPGLDHQDLMPAVGVPEALPTYEERLARA
LGEPVLPLGMPFDLRYAGPLSVEAQRDPALRTGSNPLWLRTNGALPGDL
PPLVHAALLTYISDILLDTVALRHGLSWADGTARPRSVDHAQWFHRPFR
ADDWLLLAQDTPVAYGGRALARAQVFTRGGDLVASAVQEGLVRLRRRPA
AEPEGPVSGPGPRTPRSPGR

The amino acid sequence of Streptomyces albireticuli Pop1 (WP_170116898.1) is as follows:

(SEQ ID NO: 5)
VEEIPSSRWDVDELYDPDPLALGKTYVRHGGFVDDVDLFDAAFFGISDA
EAARMDPQQRLLLQTVWHALEHAGQNPDEIRGSDTGVFLAMMNSNNYAF
LKHDAGGLTGITAYDSMADEISISAGRIAHFLDLKGPCLTVDTACSGSL
TALHLARQSILTGECDSAVVAGVNLILSPDVHVSFCKLGLFSRAGQCRA
FDAKADGYVRSEGCVAALVRRESLAEERGDPILASVLGTAINHDGHTPA
LTAPNGRTQEQVIRTVLSRTGVDPAGVGYVEAHGTGTPVGDPIEMNAIA
GAYGHARTADRPLYVGSVKSNFGHTEAAAGLLGVIKAALSLHHETIYPS
LHLDRLNPKIDLKGAAVEVPGEPVPWPRGDTPRLAAVNSFGYSGTNAHA
ILREAPRARLGAGDTARPRPAELLVLSAKSPESLDGLADRWADYLSRAD
QETLPAAVFTAAGRAAHRHRLAVTGRGALGIANDLRLWRTRRTPPSVLS
GHPAKPARTAFVFTGQGVQYPGMSRELHDSEPVFADAVERCAEVLDTEL
PVPLRRLLFEEPSPEVLDDTRLAQPALFAVEYGLATLLRSWGVVPDAVV
GHSIGEVVAACVAGMLPLEDAARFSALRGRLMGELPRDGVMLAVAAPPE
TVRGWVSGREADVSVAAVNGPRAVVVSGRAEAVDEVARLAGAAGVRTTR
LRTSHAFHSPLMDPALPELGKAAAALRPAAPVLPVLSNVTGEPLTGAEG
PEYWSQQLRRPVLFHDSMRAVAALDCTVVVEIGPHPALRAHIPEAFGAT
GVTVIPTLSRDRKDVRNLLAAAGALFTAGAAIDLPALYRGPRHRRTSSA
PLYPFRRDRYWLTDTPDAGRREPAEPAPRRRSPAPEAPAEPEPAARIVH
RHEVRAGTPWVDHRILGSTVFPATAYLGLAVDAYASVNGHGSAPVELTD
VGFVRPLLLAPTGTSSVQIGLEGDGPATDGRFRFAVAGGEGTPRYCQGK
VGPAPRQDSAATRPEELRAAMPTELAPGRLYGLLREDGMEYGASFSTVR
EVWLDEAAGQALGRITAAPDGASRVGHEHGFATMLDGCLHLTAAAARDG
AAKGTYIPVGVGRMVLRGALPDQVWGHVRLRTNDSGTAFTARLRVLDDT
GNILAEMEDVEFRRVASLTDTSAVPAAPAGDRARESGDSRRELRERIEP
LTAEERRQAVIGWLTDEIIDTLGRMSAELAVDIHHLDPSLALLEIGLDS
LSITELQRRIQEKLDFRFKAMEALEYQSIEELAEYLVQRVILAEPADAA
TAPTDS

The amino acid sequence of Streptomyces albireticuli Pop2 (PAU45553.1) is as follows:

(SEQ ID NO: 6)
VLTPDFVNNYLDSHLAERQVNKIQHGFPSPRFWNE
LSVPLDEIGEDRRRLSETHNESPVFLYIGVPYCIK
TDPGKCGYCLFPVEEFQGNAALENYYGYVEREAEM
YREQMEGVLLAGAYFGGGTSNLYRPAVYHRIMDMV
RRLFPEISDQADLTLEGIPQLFTREKMRAIADSGM
NRISMGVQQINERLNSFSGRKQTTKHVIQSLEWAR
ELGLAANVDLIFGWPQQTVDTLLEDLETLVSWDVY
DITHYELNVGGPTDFALNRYHELPSTLANLELYRA
GRDFLVDHGYEQLSTYNFRRPGDPTTRDFREGYTT
RFDHVDSLGLGYAAITFFGNPALPSGRSWSFINHR
SLPQYKAAIDNGRFPVERGFRHTPDDWLLMLLFRS
LISTDIDRTRYRTALGLDIYEKFATIWDALAERGL
AKVTPERIKLVDDGAFYAPMISALVAEERYRELRE
QAARHRRESRGHAAAAGVTLPVPGVGSGG

The amino acid sequence of Streptomyces albireticuli Pop3 (WP_095583911.1) is as follows:

(SEQ ID NO: 7)
VADDLPLAGRGVIVTGGSRGIGAAVVRLALAQGAD
VVFGYHSGEDRARALADELRAAHPGQRCTPLYAHV
ADAGEAERFAGAALDRLDRFDVLVNNAGVTRDTLF
ARMAPQQWHEVIATNLDSMYTVTKPLLMPLVKQHS
GAIVNIASSSGLHGIPGQTAYSAAKAGVIGFTKAL
AKEIGARGVTVNAVAPGLIETDMTAAIPEDKAEFL
KSLIPGHAFGSPEDVAHLVCFLASDRARYITGQAV
EVSGGLVV
>pop4 Streptomyces albireticuli
(WP_095583912.1)
(SEQ ID NO: 8)
MDRERALLDLLDLAPGGGTPGRGEGVHFVGRPPLE
QSVPVYGGHLAAQALAAAGRTVPAALPAHSVHCSF
LRPTLPSAPFEYRVEKVRNSASFATRRVHATQHGL
EVFDLTASFHRPGPGLDHQDPMPPVPDPESLPTYE
ERLTTAFGEVMQPLGKPYELRFVGPLSFDTEKNPS
LSSPRTRVWVRAEGELPDETAAGGARLLHACLLVY
VCDVTMLETVLVRHGISWFHADGRSVDYTVWIHRP
FRADDWLLCALETPAASGGRGLVLGRVFTRAGVLV
ATLAQEGLIRVSAGHGALG

The amino acid sequence of Streptomyces albireticuli Pop5 (PAU45556.1) is as follows:

(SEQ ID NO: 9)
MSDRFSAALYRRRTTVLWVSALALVLAALGGLGVE
NRLAHGGFSDPHAESSRAGRLVSEHFPTTDGDLIL
LLSGAGPVDSPTTASLGTDLTRRAERAAGVRAAGS
YWTAGRPSALRSRDGSMGLVSLSLSGDEHAQAKTA
ERLVPELRRHARGLTVMAAGPAQVQTEVGKQTAHD
LLLAEAIAMPITLVLLLLIFGSAVAAALPLVIALL
SVLVSRAVLNALAGVVSISVYSMNSTTALGLGLGI
DYSLFMLSRFREELRGGATVREALGPTVRRAGRTV
AFSGLTVALSLVALLVFPQYFLRSFAYGGIVVVLS
AAAGAVFVLPALLAVLGHRVDRYDVFARLRGPARR
ATSGTAAPVSPENGRWYRFAMAVMRRPLLYGGGAV
IVLVVLASPFTRVSSGLFDDRSLPVDSQVHRATRL
LRERFDRDVLRTVPVVVEGVGQSGRQALEPYARAL
SEVRDVRQVSAAPGAYAAGRQVRGPGAAGAALVDG
DTALFSVVSAVEQDSSAGTRLVDRLRKVAPPEGST
VSVGGRAAEVRDSTSAIARATPAAIGIVVGSSLVL
LFLFTGSVLMPVKALVLNTFSLSATFGAMVFVFQE
GHLSPLVGSPTHTGTLDATIPILTFCVAFGLSMDY
EVFLLSRIRERYLRTGDNRESVAFGLQHTGRIITA
AALLVAVVLFVFAVSGVTLLKLLGVGLALAVVLDA
TLVRALLVPSFMRLAGRANWWAPGPLRRLHNRVGL
REDADG

The amino acid sequence of Streptomyces coelicolor SCO1476 MetK (WP_003977350.1) is as follows:

(SEQ ID NO: 10)
VSRRLFTSESVTEGHPDKIADQISDTILDALLRED
PTSRVAVETLITTGLVHVAGEVTTKAYADIANLVR
GKILEIGYDSSKKGFDGASCGVSVSIGAQSPDIAQ
GVDTAYENRVEGDEDELDRQGAGDQGLMFGYASDE
TPTLMPLPVFLAHRLSKRLSEVRKNGTIPYLRPDG
KTQVTIEYDGDKAVRLDTVVVSSQHASDIDLESLL
APDIKEFVVEPELKALLEDGIKIDTENYRLLVNPT
GRFEIGGPMGDAGLTGRKIIIDTYGGMARHGGGAF
SGKDPSKVDRSAAYAMRWVAKNVVAAGLAARCEVQ
VAYAIGKAEPVGLFVETFGTAKVDTEKIEKAIDEV
FDLRPAAIIRALDLLRPIYAQTAAYGHFGRELPDF
TWERTDRVDALREAAGL

The amino acid sequence of Streptomyces coelicolor SCO6196 FadD1 (WP_011030732.1) is as follows:

(SEQ ID NO: 11)
VTAPAPQPSYAHGTSTTPLLGDTVGANLGRAIAAH
PDREALVDVPSGRRWTYAEFGAAVDELARGLLAKG
VTRGDRVGIWAVNCPEWVLVQYATARIGVIMVNVN
PAYRAHELEYVLQQSGISLLVASLAHKSSDYRAIV
EQVRGRCPALRETVYIGDPSWDALTAGAAAVEQDR
VDALAAELSCDDPVNIQYTSGTTGFPKGATLSHHN
ILNNGYWVGRTVGYTEQDRVCLPVPFYHCFGMVMG
NLGATSHGACIVIPAPSSEPAATLEAVQRERCTSL
YGVPTMFIAELNLPDFASYDLTSLRTGIMAGSPCP
VEVMKRVVAEMHMEQVSICYGMTETSPVSLQTRMD
DDLEHRTGTVGRVLPHIEVKVVDPVTGVTLPRGEA
GELRTRGYSVMLGYWEEPGKTAEAIDPGRWMHTGD
LAVMREDGYVEIVGRIKDMIIRGGENIYPREVEEF
LYAHPKIADVQVVGVPHERYGEEVLACVVVRDAAD
PLTLEELRAYCAGQLAHYKVPSRLQLLDSFPMTVS
GKVRKVELRERYGARP

The amino acid sequence of Streptomyces albireticuli ORF 1973 (WP_095580559.1) is as follows:

(SEQ ID NO: 12)
MDEGGGVPGRAEASVPRILGRDPLPGGWVRGGPPQ
VWLLRIADHAPEPPEVYERILDADERGRATAFFRD
LHRERYTAAHLGLRRLLGAYLGTGPADVALIREPC
PGCGKPHGRPAVAGAPLHFNLSHAGDLAFFAFADT
PVGADVEEEQPAEVVDGVVRMLHPDETAEIGALPG
PDRAAAFARCWTRKEAYLKGTGTGLSESPAVTYVG
SGAAPVSPPGWTLTDVAVGAGHAAAIAVATA

The amino acid sequence of Streptomyces albireticuli ORF 2980 (WP_095580559.1) is as follows:

(SEQ ID NO: 13)
MDEGGGVPGRAEASVPRILGRDPLPGGWVRGGPPQ
VWLLRIADHAPEPPEVYERILDADERGRATAFFRD
LHRERYTAAHLGLRRLLGAYLGTGPADVALIREPC
PGCGKPHGRPAVAGAPLHFNLSHAGDLAFFAFADT
PVGADVEEEQPAEVVDGVVRMLHPDETAEIGALPG
PDRAAAFARCWTRKEAYLKGTGTGLSESPAVTYVG
SGAAPVSPPGWTLTDVAVGAGHAAAIAVATA

The amino acid sequence of Streptomyces coelicolor SC03798 PirA (WP_003975143.1) is as follows:

(SEQ ID NO: 14)
MPAVTVENPLTLPRVSAPADAVARPVLTVTTAPSG
FEGEGFPVRRAFAGINYRHLDPFIMMDQMGEVEYA
PGEPKGTPWHPHRGFETVTYIVDGIFDHQDSNGGG
GTITNGDTQWMTAGSGLLHIEAPPEQLVMSGGLFH
GLQLWVNLPAKDKMMAPRYQDIRSGSVQLLTSPDG
GALLRVIAGELDGHDGPGITHTPITMVHATLAPGA
EVTLPWREDFNGLAYVMAGRGSVGAERRPVHLGQT
AVFGAGGSLTVRADEKQDAHTPDLEVVLLGGRPIR
EPMAHYGPFVMNTKDELMQAFEDFQKGRLGTVPAV
HGMSGEGPGA

In some embodiments, the O-methyltransferase is Drosophila melanogaster juvenile hormone acid methyl transferase (NP_001285980.1), or a homologous enzyme thereof. The amino acid sequence of Drosophila melanogaster juvenile hormone acid methyl transferase (NP_001285980.1) is as follows:

(SEQ ID NO: 15)
MNQASLYQHANQVQRHDAKLILDEFASTMQWRSDGEDALLDVGSGSGNVL
MDFVKPLLPIRGQLVGTDISSQMVHYASKHYQREERTRFQVLDIGCERLP
EELSGRFDHVTSFYCLHWVQNLKGALGNIYNLLKPEGGDCLLAFLASNPV
YEVYKILKTNDKWSTFMQDVENFISPLHYSLSPGEEFSQLLNDVGFVQHN
VEIRNEVFVYEGVRTLKDNVKAICPFLERMPADLHEQFLDDFIDIVISMN
LQQGENNEDQKFLSPYKLVVAYARKTPEFVNNVFLEPTHQNLVKGIN

In some embodiments, the decarbonylase is Synechococcus elongatus PCC7942 ORF594 (Q54765.1), or a homologous enzyme thereof. The amino acid sequence of Synechococcus elongatus PCC7942 ORF594 (Q54765.1) is as follows:

(SEQ ID NO: 16)
MFGLIGHLTSLEQARDVSRRMGYDEYADQGLEFWSSAPPQIVDEITVTSA
TGKVIHGRYIESCFLPEMLAARRFKTATRKVLNAMSHAQKHGIDISALGG
FTSIIFENFDLASLRQVRDTTLEFERFTTGNTHTAYVICRQVEAAAKTLG
IDITQATVAVVGATGDIGSAVCRWLDLKLGVGDLILTARNQERLDNLQAE
LGRGKILPLEAALPEADFIVWVASMPQGVVIDPATLKQPCVLIDGGYPKN
LGSKVQGEGIYVLNGGVVEHCFDIDWQIMSAAEMARPERQMFACFAEAML
LEFEGWHTNFSWGRNQITIEKMEAIGEASVRHGFQPLALAI

In some embodiments, the decarbonylase is Synechococcus elongatus PCC7942 ORF593 (Q54764.1), or a homologous enzyme thereof. The amino acid sequence of Synechococcus elongatus PCC7942 ORF593 (Q54764.1) is as follows:

(SEQ ID NO: 17)
MPQLEASLELDFQSESYKDAYSRINAIVIEGEQEAFDNYNRLAEMLPDQR
DELHKLAKMEQRHMKGFMACGKNLSVTPDMGFAQKFFERLHENFKAAAAE
GKVVTCLLIQSLIIECFAIAAYNIYIPVADAFARKITEGVVRDEYLHRNF
GEEWLKANFDASKAELEEANRQNLPLVWLMLNEVADDARELGMERESLVE
DFMIAYGEALENIGFTTREIMRMSAYGLAAV

The amino acid sequence of Streptomyces globisporus SGCE10 (WP_010056311.1) is as follows:

(SEQ ID NO: 18)
SNAMTATNPDYFELRHTVGFEETNLVGNVYYVNYLRWQGRCRELFLKERA
PSVLAEVQEDLKLFTLKVDCEFFAEITAFDELSIRMRLSELRQTQLEFTF
DYIKLGDDGGETLVARGRQRIACMRGPNTATVPTLIPEALAEALAPYSDR
AGSYAGRAA

In some embodiments, the decarboxylase is Jeotgalicoccus sp. ATCC8456 OleT, or a homologous enzyme thereof. The amino acid sequence of Jeotgalicoccus sp. ATCC8456 OleT (ADW41779.1) is as follows:

(SEQ ID NO: 19)
MATLKRDKGLDNTLKVLKQGYLYTTNQRNRLNTSVFQTKALGGKPFVVVT
GKEGAEMFYNNDVVQREGMLPKRIVNTLFGKGAIHTVDGKKHVDRKALFM
SLMTEGNLNYVRELTRTLWHANTQRMESMDEVNIYRESIVLLTKVGTRWA
GVQAPPEDIERIATDMDIMIDSFRALGGAFKGYKASKEARRRVEDWLEEQ
IIETRKGNIHPPEGTALYEFAHWEDYLGNPMDSRTCAIDLMNTFRPLIAI
NRFVSFGLHAMNENPITREKIKSEPDYAYKFAQEVRRYYPFVPFLPGKAK
VDIDFQGVTIPAGVGLALDVYGTTHDESLWDDPNEFRPERFETWDGSPFD
LIPQGGGDYWTNHRCAGEWITVIIMEETMKYFAEKITYDVPEQDLEVDLN
SIPGYVKSGFVIKNVREVVDRT

In some embodiments, the O-methyltransferase is Mycobacterium marinum fatty acid O-methyltransferase (WP_012395002.1), or a homologous enzyme thereof. The amino acid sequence of Mycobacterium marinum fatty acid O-methyltransferase (WP_012395002.1) is as follows:

(SEQ ID NO: 20)
MPREIRLPESSVVVRPAPMESATYSQSSRLQAAGLSPAITLFEKAAQTVP
LPDAPQPVVIADYGVATGHNSLKPMMAAINALRRRIREDRAIMVAHTDVP
DNDFTALFRTLADDPDSYLHHDSASFASAVGRSFYTQILPSNTVSLGWSS
WAIQWLSRIPAGAPELTDHVQVAYSKDERARAAYAHQAATDWQDFLAFRG
RELCPGGRLVVLTMALDEHGHFGYRPMNDALVAALNDQVRDGLLRPEELR
RMAIPVVARAEKDLRAPFAPRGWFEGLTIEQLDVFNAEDRFWAAFQSDGD
AESFGAQWAGFARAALFPTLAAALDCGTGDPRATAFIEQLEASVADRLAS
QPEPMRIPLASLVLAKRA

In some embodiments, the ethyl transferase is Acinetobacter baylyi ADP1 wax-ester synthase (WP_004922247.1), or a homologous enzyme thereof. The amino acid sequence of Acinetobacter baylyi ADP1 wax-ester synthase (WP_004922247.1) is as follows:

(SEQ ID NO: 21)
MRPLHPIDFIFLSLEKRQQPMHVGGLFLFQIPDNAPDTFIQDLVNDIRIS
KSIPVPPFNNKLNGLFWDEDEEFDLDHHFRHIALPHPGRIRELLIYISQE
HSTLLDRAKPLWTCNIIEGIEGNRFAMYFKIHHAMVDGVAGMRLIEKSLS
HDVTEKSIVPPWCVEGKRAKRLREPKTGKIKKIMSGIKSQLQATPTVIQE
LSQTVFKDIGRNPDHVSSFQAPCSILNQRVSSSRRFAAQSFDLDRFRNIA
KSLNVTINDVVLAVCSGALRAYLMSHNSLPSKPLIAMVPASIRNDDSDVS
NRITMILANLATHKDDPLQRLEIIRRSVQNSKQRFKRMTSDQILNYSAVV
YGPAGLNIISGMMPKRQAFNLVISNVPGPREPLYWNGAKLDALYPASIVL
DGQALNITMTSYLDKLEVGLIACRNALPRMQNLLTHLEEEIQLFEGVIAK
QEDIKTAN

In some embodiments, the host cell comprises a nucleic acid encoding the one or more enzymes operatively linked to a promoter capable of expressing the one or more enzymes in the host cell. In some embodiments, the encoding of the one or more enzymes to the nucleic acid is codon optimized to the host cell. In some embodiments, the nucleic acid is vector or replicon that can stably reside in the host cell. In some embodiments, the nucleic acid is stably integrated into one or more chromosomes of the host cell.

In some embodiments, the providing step (a) comprises introducing a nucleic acid encoding the one or more enzymes operatively linked to a promoter capable of expressing the one or more enzymes in the host cell into the host cell.

In some embodiments, the culturing or growing step (b) comprises the host cell growing by respiratory cell growth. In some embodiments, the culturing or growing step (b) takes place in a batch process or a fed-batch process, such as a high-gravity fed-batch process. In some embodiments, the culture or medium comprises hydrolysates derived or obtained from a biomass, such as a lignocellulosic biomass. In some embodiments, the culture or medium comprises one or more carbon sources, such as a sugar, such as glucose or galactose, or glycerol, or a mixture thereof. In some embodiments, the carbon source is fermentable. In some embodiments, the carbon source is non-fermentable.

The present invention provides for a method for constructing a genetically modified host cell of the present invention, comprising (a) introducing a nucleic acid encoding the one or more enzymes operatively linked to a promoter capable of expressing the one or more enzymes in the host cell into the host cell.

One can modify the expression of a gene encoding any of the enzymes taught herein by a variety of methods in accordance with the methods of the invention. Those skilled in the art would recognize that increasing gene copy number, ribosome binding site strength, promoter strength, and various transcriptional regulators can be employed to alter an enzyme expression level.

### Suitable Biomass

The biomass can be obtained from one or more feedstock, such as softwood feedstock, hardwood feedstock, grass feedstock, and/or agricultural feedstock, or a mixture thereof.

Softwood feedstocks include, but are not limited to, Araucaria (e.g. A. cunninghamii, A. angustifolia, A. araucana); softwood Cedar (e.g. Juniperus virginiana, Thuja plicata, Thuja occidentalis, Chamaecyparis thyoides Callitropsis nootkatensis); Cypress (e.g. Chamaecyparis, Cupressus Taxodium, Cupressus arizonica, Taxodium distichum, Chamaecyparis obtusa, Chamaecyparis lawsoniana, Cupressus semperviren); Rocky Mountain Douglas fir; European Yew; Fir (e.g. Abies balsamea, Abies alba, Abies procera, Abies amabilis); Hemlock (e.g. Tsuga canadensis, Tsuga mertensiana, Tsuga heterophylla); Kauri; Kaya; Larch (e.g. Larix decidua, Larix kaempferi, Larix laricina, Larix occidentalis); Pine (e.g. Pinus nigra, Pinus banksiana, Pinus contorta, Pinus radiata, Pinus ponderosa, Pinus resinosa, Pinus sylvestris, Pinus strobus, Pinus monticola, Pinus lambertiana, Pinus taeda, Pinus palustris, Pinus rigida, Pinus echinata); Redwood; Rimu; Spruce (e.g. Picea abies, Picea mariana, Picea rubens, Picea sitchensis, Picea glauca); Sugi; and combinations/hybrids thereof.

For example, softwood feedstocks which may be used herein include cedar; fir; pine; spruce; and combinations thereof. The softwood feedstocks for the present invention may be selected from loblolly pine (Pinus taeda), radiata pine, jack pine, spruce (e.g., white, interior, black), Douglas fir, Pinus silvestris, Picea abies, and combinations/hybrids thereof. The softwood feedstocks for the present invention may be selected from pine (e.g. Pinus radiata, Pinus taeda); spruce; and combinations/hybrids thereof.

Hardwood feedstocks include, but are not limited to, Acacia; Afzelia; Synsepalum duloificum; Albizia; Alder (e.g. Alnus glutinosa, Alnus rubra); Applewood; Arbutus; Ash (e.g. F. nigra, F. quadrangulata, F. excelsior, F. pennsylvanica lanceolata, F. latifolia, F. profunda, F. americana); Aspen (e.g. P. grandidentata, P. tremula, P. tremuloides); Australian Red Cedar (Toona ciliata); Ayna (Distemonanthus benthamianus); Balsa (Ochroma pyramidale); Basswood (e.g. T. americana, T. heterophylla); Beech (e.g. F. sylvatica, F. grandifolia); Birch; (e.g. Betula populifolia, B. nigra, B. papyrifera, B. lenta, B. alleghaniensis/B. lutea, B. pendula, B. pubescens); Blackbean; Blackwood; Bocote; Boxelder; Boxwood; Brazilwood; Bubing a; Buckeye (e.g. Aesculus hippocastanum, Aesculus glabra, Aesculus flava/Aesculus octandra); Butternut; Catalpa; Chemy (e.g. Prunus serotina, Prunus pennsylvanica, Prunus avium); Crabwood; Chestnut; Coachwood; Cocobolo; Corkwood; Cottonwood (e.g. Populus balsamifera, Populus deltoides, Populus sargentii, Populus heterophylla); Cucumbertree; Dogwood (e.g. Cornus florida, Cornus nuttallii); Ebony (e.g. Diospyros kurzii, Diospyros melanida, Diospyros crassiflora); Elm (e.g. Ulmus americana, Ulmus procera, Ulmus thomasii, Ulmus rubra, Ulmus glabra); Eucalyptus; Greenheart; Grenadilla; Gum (e.g. Nyssa sylvatica, Eucalyptus globulus, Liquidambar styraciflua, Nyssa aquatica); Hickory (e.g. Carya alba, Carya glabra, Carya ovata, Carya laciniosa); Hornbeam; Hophornbeam; Ip6; Iroko; Ironwood (e.g. Bangkirai, Carpinus caroliniana, Casuarina equisetifolia, Choricbangarpia subargentea, Copaifera spp., Eusideroxylon zwageri, Guajacum officinale, Guajacum sanctum, Hopea odorata, Ipe, Krugiodendronferreum, Lyonothamnus lyonii (L. floribundus), Mesua ferrea, Olea spp., Olneya tesota, Ostrya virginiana, Parrotia persica, Tabebuia serratifolia); Jacarandi; Jotoba; Lacewood; Laurel; Limba; Lignum vitae; Locust (e.g. Robinia pseudacacia, Gleditsia triacanthos); Mahogany; Maple (e.g. Acer saccharum, Acer nigrum, Acer negundo, Acer rubrum, Acer saccharinum, Acer pseudoplatanus); Meranti; Mpingo; Oak (e.g. Quercus macrocarpa, Quercus alba, Quercus stellata, Quercus bicolor, Quercus virginiana, Quercus michauxii, Quercus prinus, Quercus muhlenbergii, Quercus chrysolepis, Quercus lyrata, Quercus robur, Quercus petraea, Quercus rubra, Quercus velutina, Quercus laurifolia, Quercus falcata, Quercus nigra, Quercus phellos, Quercus texana); Obeche; Okoume; Oregon Myrtle; California Bay Laurel; Pear; Poplar (e.g. P. balsamifera, P. nigra, Hybrid Poplar (Populus×canadensis)); Ramin; Red cedar; Rosewood; Sal; Sandalwood; Sassafras; Satinwood; Silky Oak; Silver Wattle; Snakewood; Sourwood; Spanish cedar; American sycamore; Teak; Walnut (e.g. Juglans nigra, Juglans regia); Willow (e.g. Salix nigra, Salix alba); Yellow poplar (Liriodendron tulipifera); Bamboo; Palmwood; and combinations/hybrids thereof.

For example, hardwood feedstocks for the present invention may be selected from Acacia, Aspen, Beech, Eucalyptus, Maple, Birch, Gum, Oak, Poplar, and combinations/hybrids thereof. The hardwood feedstocks for the present invention may be selected from Populus spp. (e.g. Populus tremuloides), Eucalyptus spp. (e.g. Eucalyptus globulus), Acacia spp. (e.g. Acacia dealbata), and combinations thereof.

Grass feedstocks include, but are not limited to, C₄ or C₃ grasses, e.g. Switchgrass, Indiangrass, Big Bluestem, Little Bluestem, Canada Wildrye, Virginia Wildrye, and Goldenrod wildflowers, etc, amongst other species known in the art.

Agricultural feedstocks include, but are not limited to, agricultural byproducts such as husks, stovers, foliage, and the like. Such agricultural byproducts can be derived from crops for human consumption, animal consumption, or other non-consumption purposes. Such crops can be corps such as corn, wheat, rice, soybeans, hay, potatoes, cotton, or sugarcane.

The feedstock can arise from the harvesting of crops from the following practices: intercropping, mixed intercropping, row cropping, relay cropping, and the like.

### Fuel Composition

In some embodiments, the fuel composition further comprises tricyclic sesquiterpene prespatane, pentalenene, α-isocomene, α-zingiberene, β-sesquiphellandrene, α-bisabolene, β-bisabolene, γ-bisabolene, curcumene, gossonorol, or any monocyclic sesquiterpene taught in U.S. Pat. No. 9,109,175 (herein incorporated by reference), or a mixture thereof.

In one embodiment, the fuel additive that is mixed with the cyclopropane compound hydrogenation is a chemical compound or component added to the fuel composition to alter the property of the fuel, e.g., to improve engine performance, fuel handling, fuel stability, or for contaminant control, etc. The nature and amount of the one or more additives depends on the desired use of the final fuel composition. Some nonlimiting examples of conventional fuel additives include antioxidants, thermal stability improvers, cetane improvers, stabilizers, cold flow improvers, combustion improvers, anti-foams, anti-haze additives, corrosion inhibitors, lubricity improvers, icing inhibitors, injector cleanliness additives, smoke suppressants, drag reducing additives, metal deactivators, dispersants, detergents, demulsifiers, dyes, markers, static dissipaters, biocides, and combinations thereof.

In one embodiment, the fuel additive that is mixed with an oxidizer, and optionally with a compound used as a rocket fuel, such as syntin, kerosene, alcohol, hydrazine, and derivatives thereof, and liquid hydrogen, and the like. Oxidizers include nitric acid, nitrogen tetroxide, liquid oxygen, and liquid fluorine. Some of the best oxidizers are liquified gases, such as oxygen and fluorine, which exist as liquids only at very low temperatures; this adds greatly to the difficulty of their use in rockets. Most fuels, with the exception of hydrogen, are liquids at ordinary temperatures. Certain propellant combinations are hypergolic; that is, they ignite spontaneously upon contact of the fuel and oxidizer. Others require an igniter to start them burning, although they will continue to burn when injected into the flame of the combustion chamber.

In some embodiments, the fuel composition of the present invention may further comprise a conventional fuel component derived from petroleum, coal, wood, or any other hydrocarbon source. Nonlimiting examples of conventional fuel components include, but are not limited to, diesel fuels, jet fuels, kerosene, gasoline, and Fischer-Tropsch derived fuels. In some embodiments, the conventional fuel component is derived from petroleum or coal. In certain embodiments, the fuel component is or comprises a diesel fuel, a jet fuel, kerosene, gasoline, or a combination thereof. In other embodiments, the fuel component is or comprises a distillate diesel fuel.

In certain embodiments, the fuel composition of the present invention is intended for use in diesel engines. In other embodiments, the fuel composition of the present invention is intended for use in jet engines and/or missile propulsion systems. As such, the fuel compositions disclosed herein can be used as a fuel for internal combustion engines such as gasoline engines, diesel engines, jet engines, and/or missile propulsion systems.

In yet another aspect, the present invention provides a vehicle comprising an internal combustion engine, a fuel tank connected to the internal combustion engine, and a fuel composition in the fuel tank, wherein the fuel composition is the fuel composition as disclosed herein (e.g., hydrogenated tricyclic sesquiterpene), wherein the fuel combustion is used to power the internal combustion engine. In one embodiment, the internal combustion engine is a diesel engine. In another embodiment, the internal combustion engine is a jet engine or missile propulsion system.

In a further aspect, the present invention provides a method of powering an engine comprising the step of combusting a fuel composition of the present invention in the engine. In one embodiment, the engine is a diesel engine. In another embodiment, the engine is a jet engine or a missile propulsion system. In another embodiment, the engine is a rocket engine or rocket propulsion system.

Other objects, features, and advantages of the present invention will be apparent to one of skill in the art from the following detailed description and figures.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

The invention having been described, the following examples are offered to illustrate the subject invention by way of illustration, not by way of limitation.

### Example 1

### Materials and Methods

To create a biosynthetic route for polycyclopropanated high-energy fuels, the biosynthetic gene cluster (BGC) for Jawsamycin is used as query. Specifically, the protein sequences of the iterative PKS, methyltransferase and ketoreductase are used as queries for genome mining across a database including about 10,000 bacterial genomes. This bioinformatic search led to the identification of 20 biosynthetic gene clusters, which are predicted will produce a polycyclopropanated product. Among the new enzymatic functions within these BGCs, a previously undescribed stand-alone (type II) thioesterase which we called pop4 is found. It is predicted that the BGCs including a pop4 ortholog will produce a polycyclopropanated fatty acid which can be modified in vivo or ex vivo to produce high energy fuels.

From the 20 BGC candidates, two previously undescribed and uncharacterized BGCs that included a pop4 ortholog are selected. The genes coding for an iterative PKS (pop1), methyl transferase (pop2), ketoreductase (pop3) and thioesterase (pop4), are cloned from Streptomyces sp. CC24 (which are isolated and which genome has not been released) and Streptomyces albireticuli into various vectors for bacterial expression in Escherichia coli, Streptomyces albus, Streptomyces coelicolor and Streptomyces lividans. The pop1-3 orthologs from Streptomyces roseoverticilatus (part of the jawsamycin BGC) together with pop4 from Streptomyces albireticuli also cloned. This is done because the Jawsamycin polyketide synthase lacks a cyclopropane acid release system and therefore cannot naturally produce cyclopropane-bearing free fatty acids. The genes are synthesized, cloned, and expressed under the control of engineered promoters for E. coli or actinobacteria to form a synthetic 4-gene operon for in vivo studies. In parallel, for in vitro studies the pop1-4 genes from Streptomyces albireticuli are codon optimized for E. coli, and independently expressed as solubility tag-fused proteins.

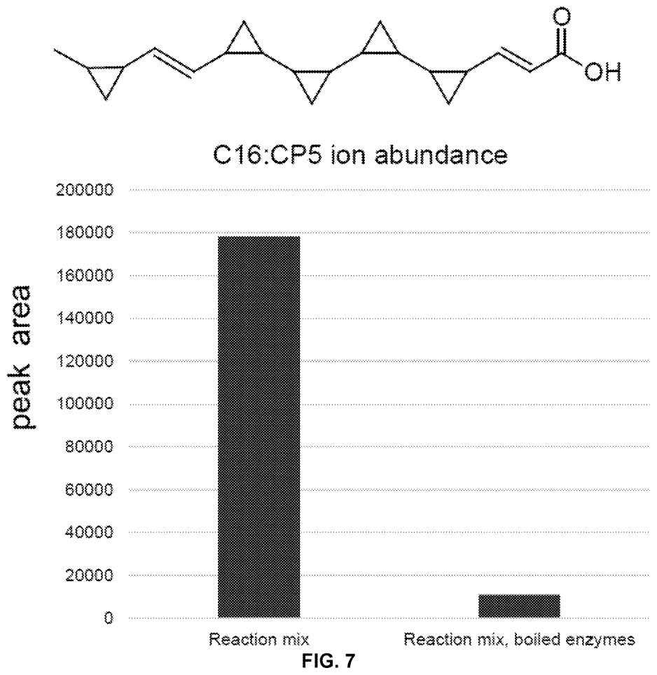

For in vitro studies, cell free extracts for each protein are mixed with His-tag purified phosphopantetheine transferase (Sfp) as well as all the substrates and cofactors needed. A product corresponding to an m/z of 311.201654 (negative mode), with retention time of 17.8 is identified, this product corresponds to a C16:CP5 fatty acid (C21H2802). FIG. 7 shows the results of an in vitro study.

For in vivo studies, the expression strains are cultivated in various media and cells harvested by centrifugation. The proteins are extracted and used for proteomic analyses to assess the expression of the heterologous genes. Peptide fragments for all proteins are identified in E. coli and Streptomyces hosts.

The pop1-4 genes from Streptomyces albireticuli and Streptomyces sp. CC24A are tested for in vivo production of polycyclopropanated acids. For this, a series of fermentations with different conditions in shake flasks are set up. The fermented cultures are then processed with various extraction methods focused in obtaining free fatty acids and in general hydrophobic molecules. The concentrated extracts are analyzed using high resolution LC-MS. These experiments showed the presence of ions corresponding to several cyclopropanated fatty acids ranging from 14 to 22 carbons in length with various cyclopropane ring compositions.

To further confirm the structure of these compounds the biosynthetic logic of the pathway is exploited: each cyclopropane ring in the products is installed by the S-Adenosyl Methionine (SAM)-dependent methyl transferase Pop2. The methylene group in each ring is therefore derived from S-adenosyl-methionine. Following this reasoning, the heterologous strain is cultivated in media supplemented with methionine (methyl-C¹³). The C¹³-labelled carbon atom in this amino acid is known to be incorporated by the host into S-Adenosyl Methionine which is in turn installed at each cyclopropane ring in the cyclopropane products. The methionine (methyl-C¹³) fed fermentations are analyzed using high resolution LC-MS. These experiments show that ions with m/z corresponding to the cyclopropane products present the predicted shift in mass corresponding to the number of cyclopropane rings upon feeding of the labeled precursor. This experiment confirms the identity of the molecules. Given the current lack of polycyclopropane acids standards for these molecules, the amount of product in the heterologous system are compared with the saturated C18:CP3 fatty acid DCPLA, at this stage the calculated production using this raw method is 1.2 mg per liter.

After confirming the identity of the molecules, focus is put on increasing the total polycyclopropane acid production for the pathway from S. albireticuli heterologously expressed into S. coelicolor. For this purpose the natural gene sequence of the pop1-4 genes is searched for TTA codons. The search confirms the presence of various TTA codons in pop1 and 2. These codons are an infrequent variant for Leucine, and its cognate tRNA (encoded in the bldA gene) is known to be expressed during late stages of growth in streptomycetes. The consequence being late expression of the genes in the heterologous pathway. To address this issue, a system for orthogonal and constitutive expression of bldA is constructed, and site directed mutagenesis is used to generate TTA-free variants of pop1 and pop2. High resolution LC-MS analysis of extracts from 50 mL cultures from these new strains shows a 60% and 100% increase in total polycyclopropanated fatty acid production correspondingly.

To further improve the production of polycyclopropanated acids, a series of strains are constructed in which: (a) the Streptomyces coelicolor gene SCO6196, a long chain acyl coA-ligase responsible for mobilization of carbon from storage lipids into polyketide biosynthesis, is overexpressed, (b) the gene SCO1476 (metK), a S-adenosyl-methionine synthase, is overexpressed, and (c) the gene SC03798 (pirA), a pirin that balances the acyl-CoA pool and buffers oxidative stress, is knocked out. PirA is the target of the phiC31 integrase which is commonly used for integration of heterologous genes in S. coelicolor, this gene is disrupted by integrating the empty plasmid using the PhC31 integrase in the heterologous producer strain. This strain yields an increase of 100% in production from the previous strain.

To increase the chances of phosphopanteteinylation of Pop1 while heterologously expressed, the specialized phosphopanteteinyl transferase orf_1973 and orf_2980 from S. albireticuli are identified, cloned, and overexpressed in S. coelicolor. Given that the polycyclopropanated acids remain inside the cells, a potential polycyclopropanated acid transporter nearby the pop BGC in Streptomyces albireticuli are searched and identified. This gene is called pop5, and is cloned and overexpressed in the heterologous system. To modify the length of the products, a series of strains are designed and constructed in which extra copies of pop4 are incorporated. The extra copies are under the control of promoters with different strengths (low, medium, and high).

To tailor the polycyclopropanated polyketides into alkanes, four different decarboxylation systems from cyanobacteria, algae, actinobacteria and firmicutes are selected. All these systems are known to take fatty acids as substrates. FAP from Chlorella variabilis NC64A, a photo-decarboxylase that leads to alkanes. OleT a cytochrome P450 decarboxylase from Jeotgalicoccus sp. ATCC 8456 that produces terminal alkenes, SgcE10 from Streptomyces sp. C-1027 that takes a polyene and generates an alkene, and Orf_593 and Orf_594 from Synechococcus sp. PCC7002, which takes an ACP-bound fatty acid and after a reductive release of an aldehyde produces an alkene. Codon optimized versions of the corresponding genes are synthesized and incorporated into the biosynthetic pathway.

For esterification of the polycyclopropanated polyketides into methyl or ethyl esters Juvenile Hormone Acid O-Methyltransferase from Drosophila melanogaster; the fatty acid methyltransferase from Mycobacterium marinum and a Wax-ester synthase from Acinetobacter baylyi ADP1 are selected.

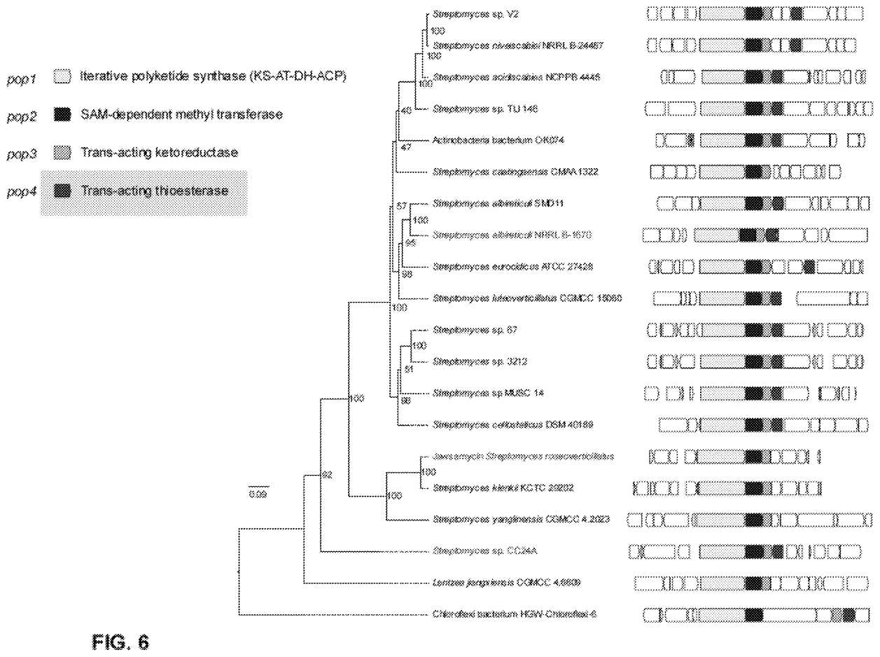

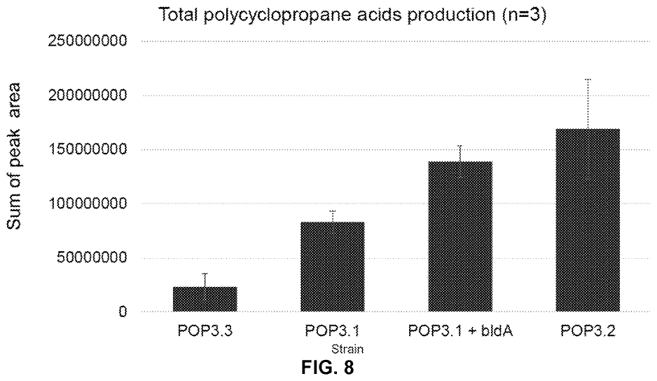

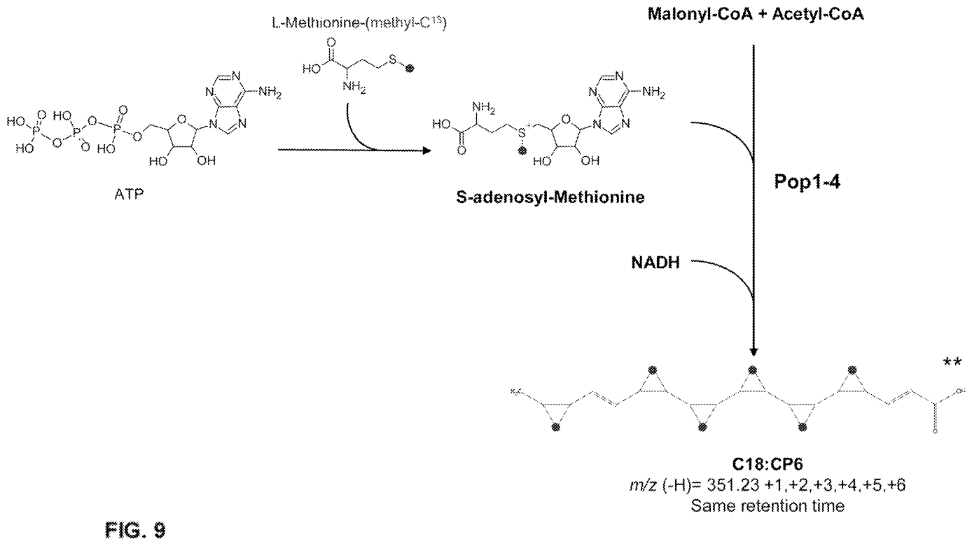

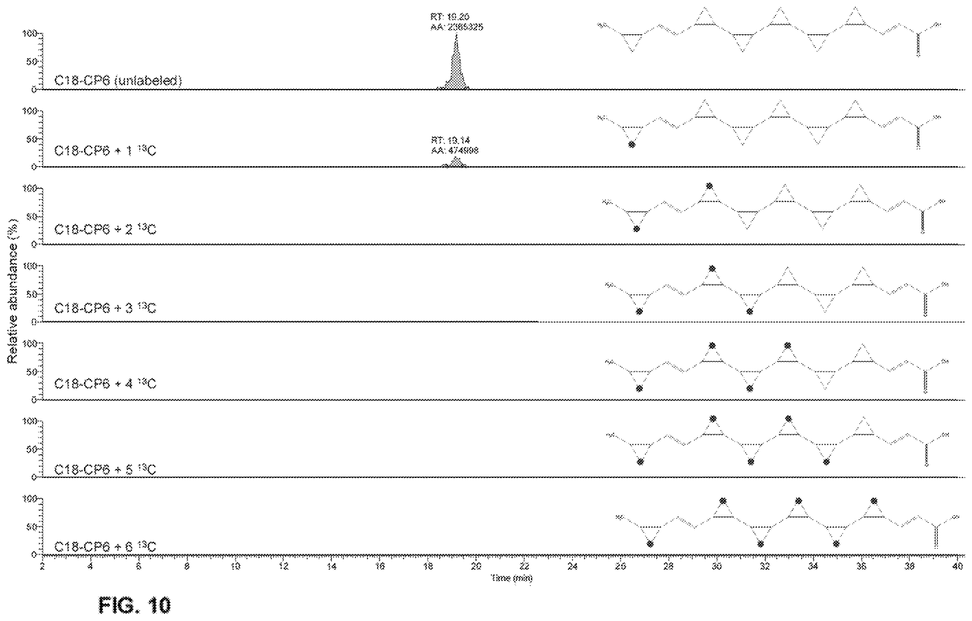

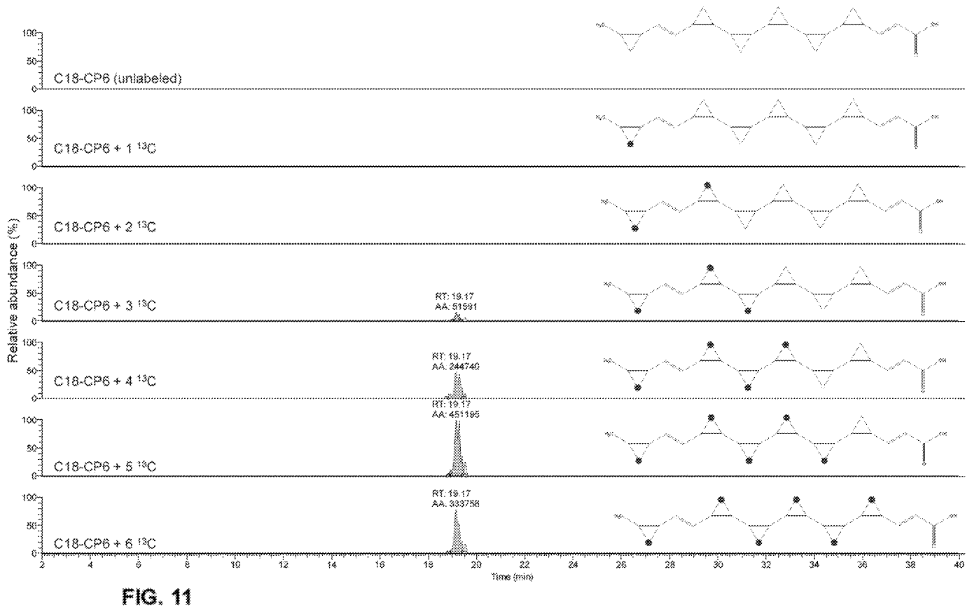

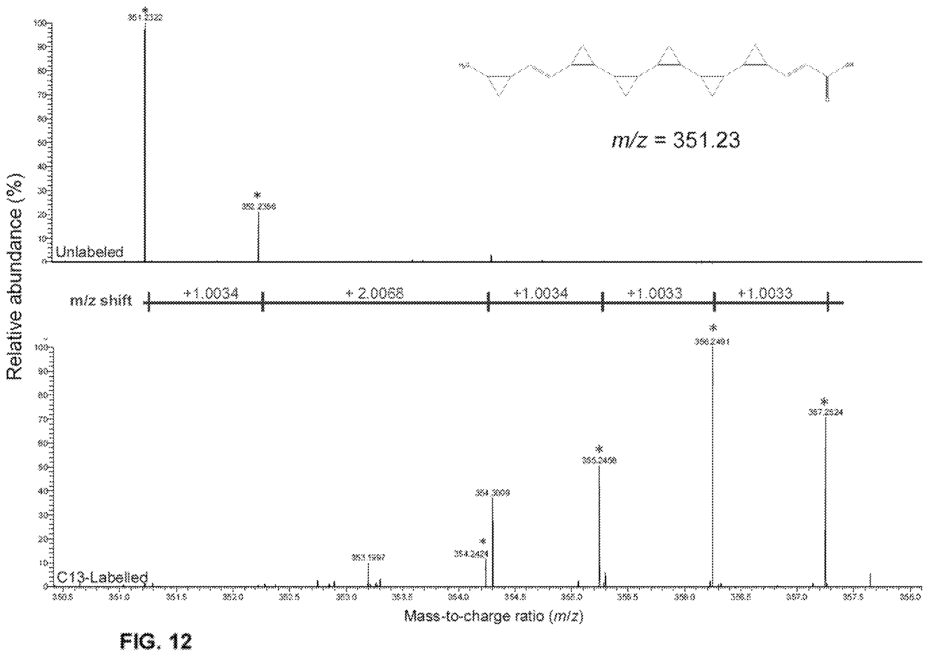

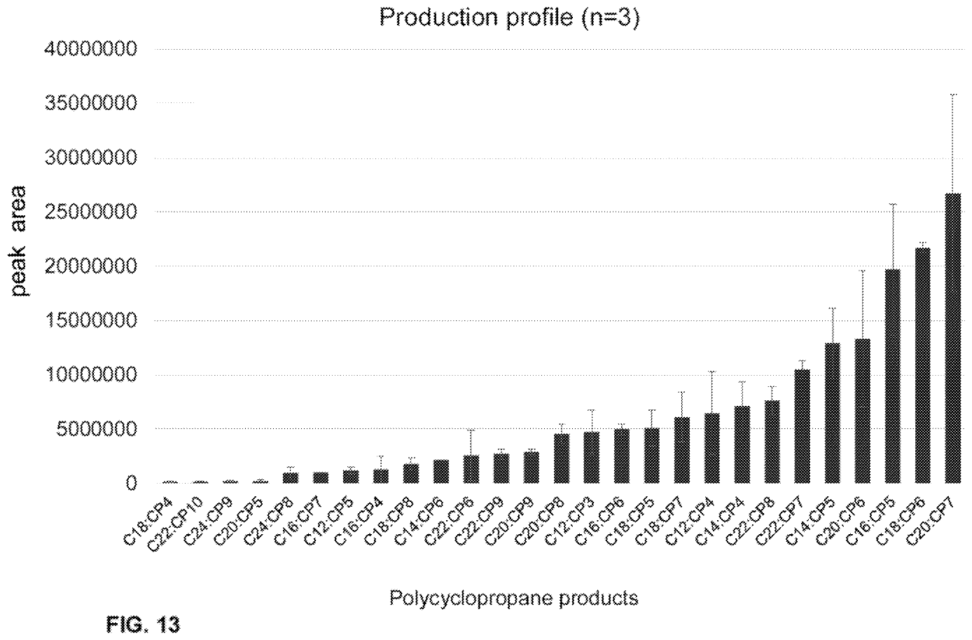

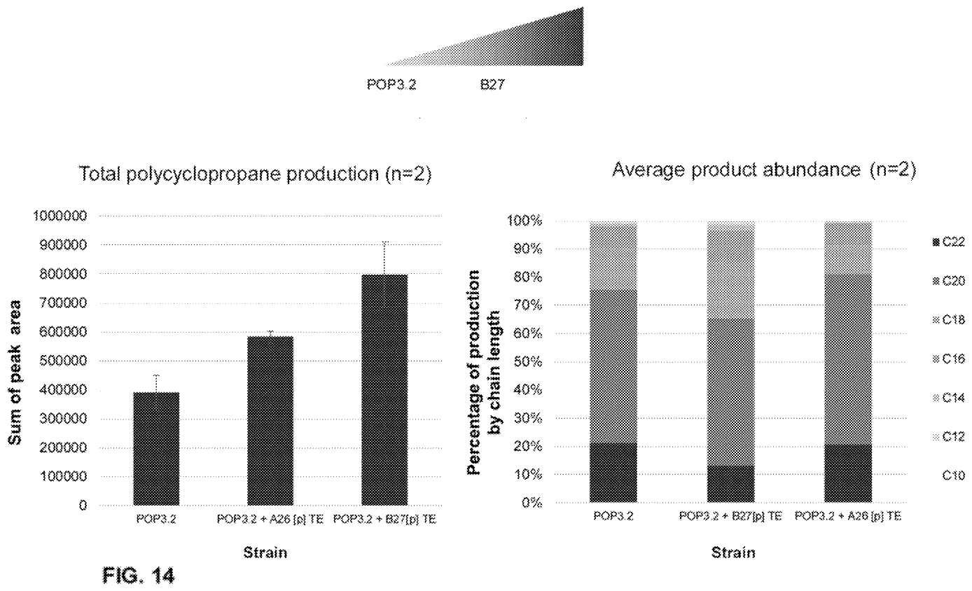

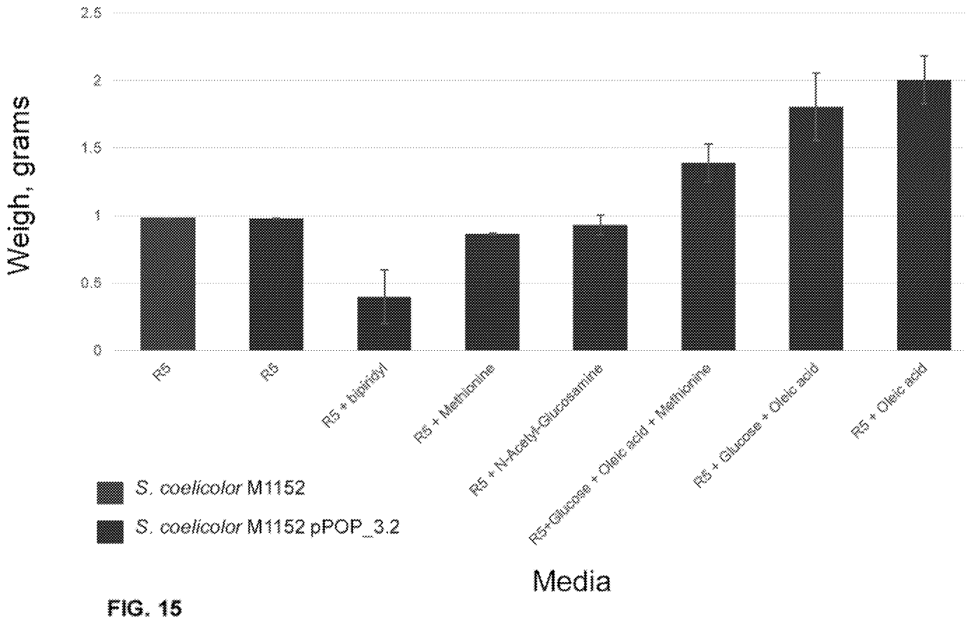

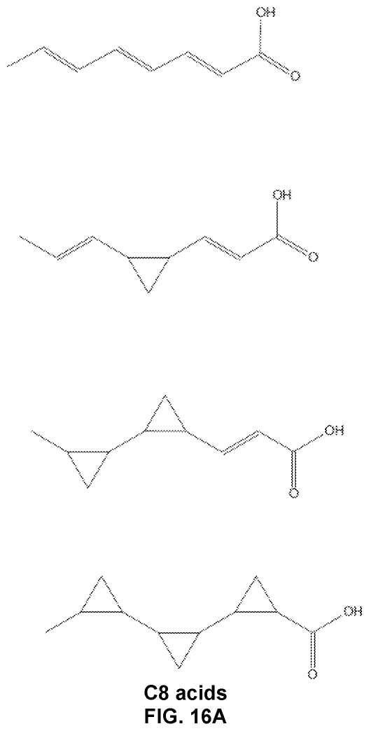

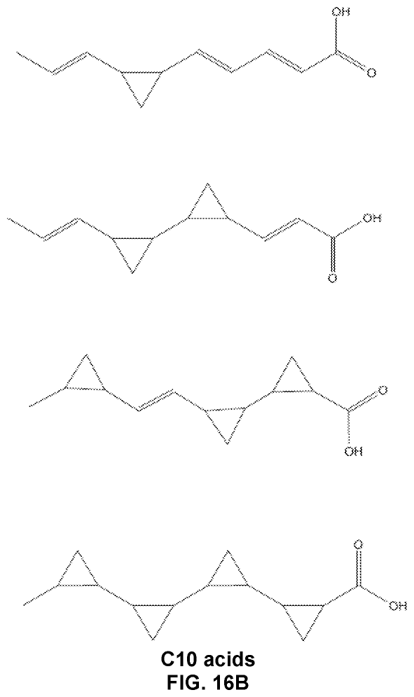

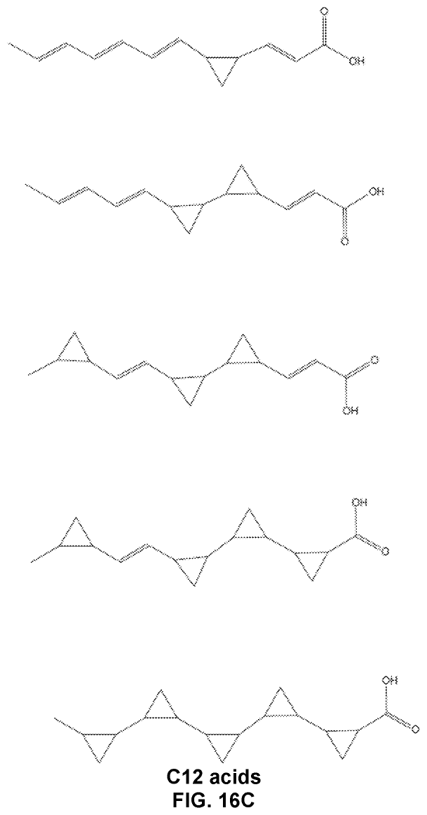

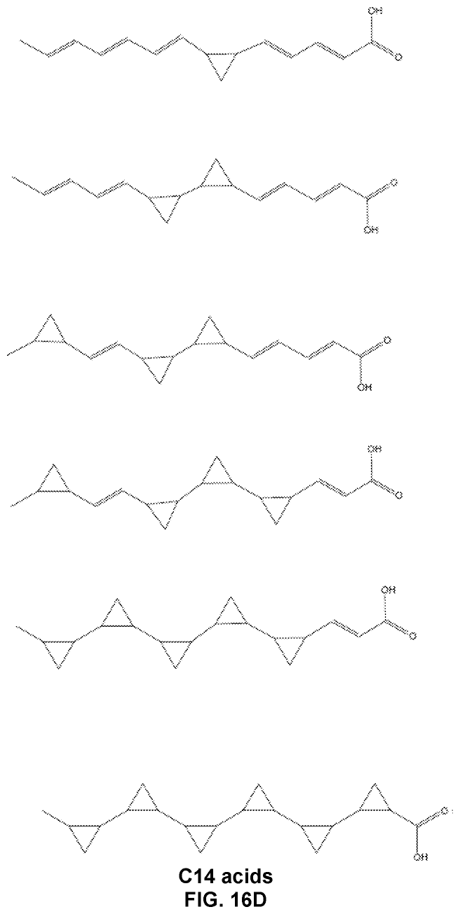

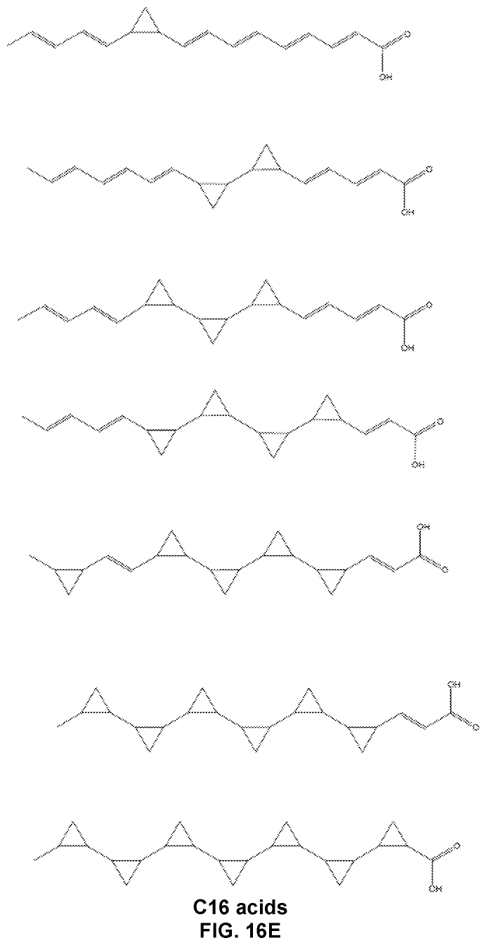

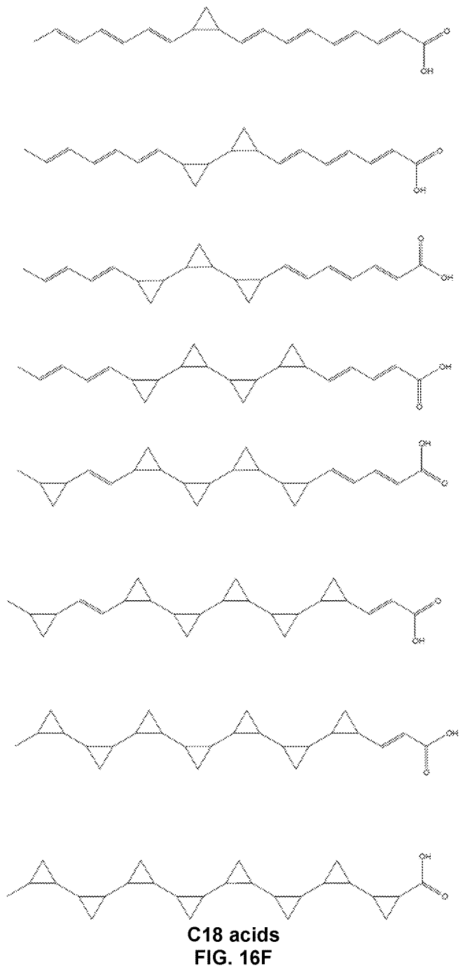

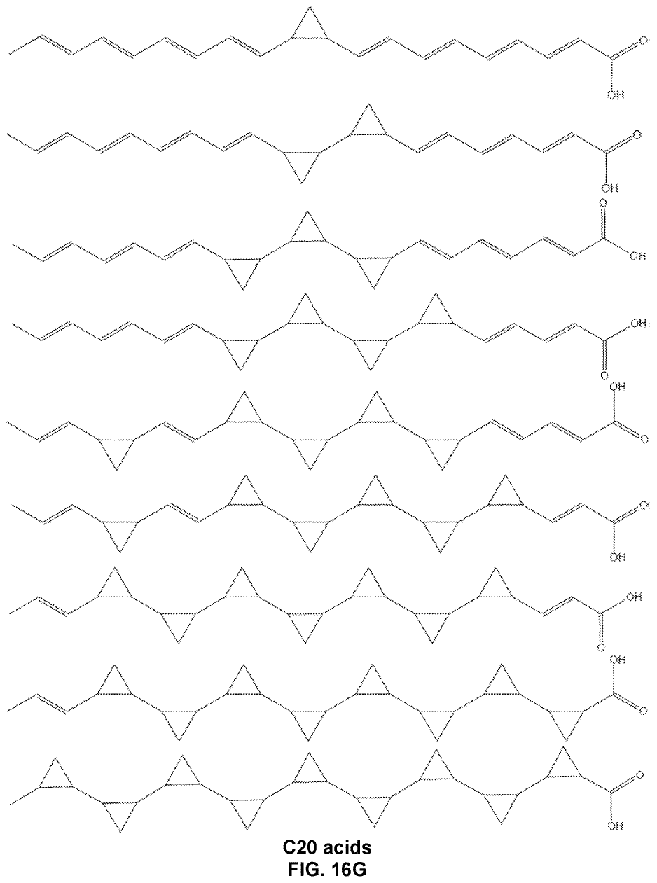

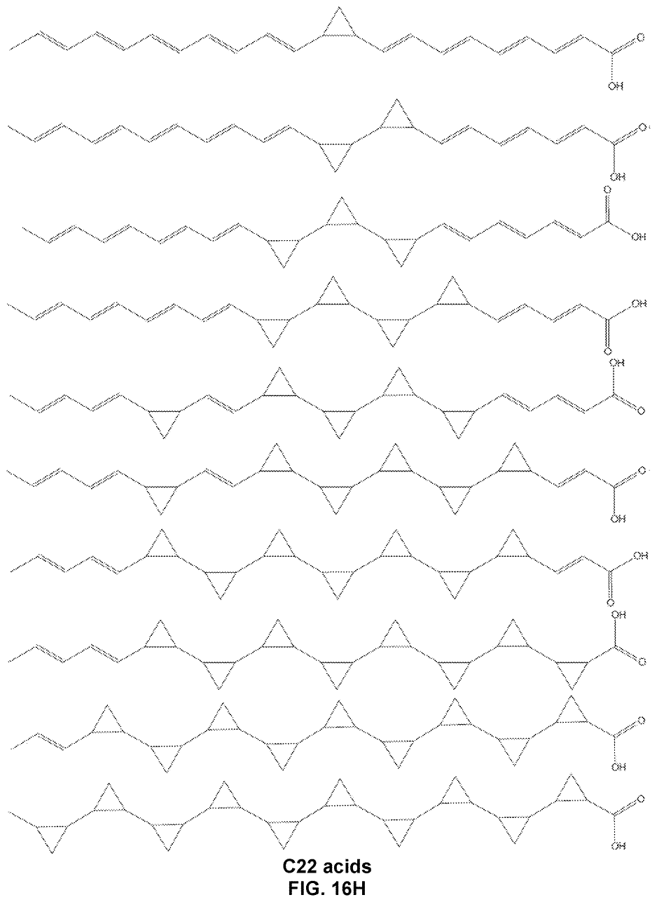

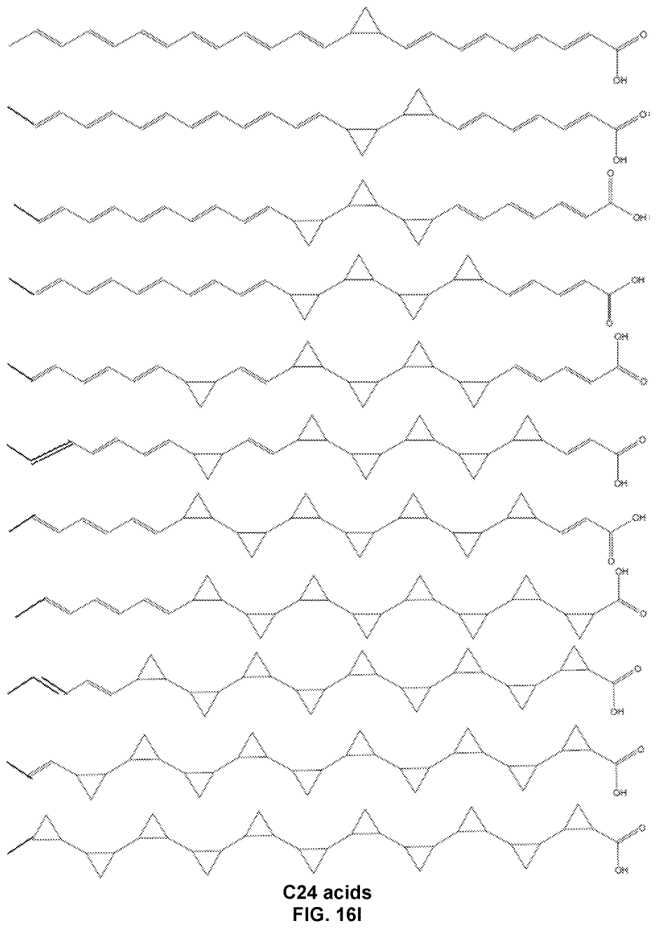

A summary of the experiments performed is shown in Table 4. The results of these experiments are shown in FIGS. 6-15.

TABLE 4
Three candidate BGCs are selected, refactored (including the
newly discovered thioesterase) and heterologously expressed
in multiple hosts.

| | Source | | | |
|---|---|---|---|---|
| | S. | S. | S. | |
| Host | roseoverticilatus | albireticuli | sp 24A | Source |
| S. coelicolor | | X | X | Yes |
| M1152 | | | | |
| S. lividans | | X | | Yes |
| TK24 | | | | |
| S. albus | | X | | No |
| J1074 | | | | |
| E. coli | X | | | No |
| BAP1 | | | | (in vivo) |
| E. coli | | X | | Yes |
| BL21 | | | | (in vitro) |

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A system comprising an iterative polyketide synthase (PKS) comprising ketosynthase (KS)-acyltransferase (AT)-dehydratase (DH)-acyl carrier protein (ACP) capable of synthesizing a cyclopropane group, a methyl transferase, a ketoreductase, and a thioesterase, which together are capable of synthesizing a cyclopropane compound having the following chemical formula: 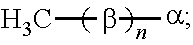
wherein α is —H or —COOR, wherein R is —H or an alkyl group, such as —CH₃, —CH₂CH₃, —(CH₂)₂—CH₃, —(CH₂)₃—CH₃, or —C(CH₃)₃; β is each independently 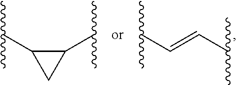
wherein at least one β is 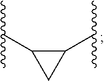 [SMILES: *C1CC1*]
and, n is an integer from 3 to 11; or a mixture thereof; with the provisio that when n=8, wherein the cyclopropane compound consists of 1, 2, 3, 4, 5, 7, or 8, 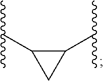 [SMILES: *C1CC1*]
and there are 1, 2, 4, 5, 6, 7, or 8,

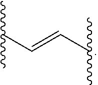 [SMILES: */C=C/*]

2. The system of claim 1, wherein the cyclopropane compound has a longest carbon chain with 12 to 24 carbon atoms.

3. The system of claim 2, wherein the cyclopropane compound has a longest carbon chain with 14 to 22 carbon atoms.

4. The system of claim 3, wherein the cyclopropane compound has a longest carbon chain with 16 to 20 carbon atoms.

5. The system of claim 1, wherein the alkyl group is —CH₃, —CH₂CH₃, —(CH₂)₂—CH₃, —(CH₂)₃—CH₃, or —C(CH₃)₃.

6. The system of claim 1, wherein the cyclopropane compound comprises or has 2, 3, 4, 5, 6, 7, 8, 9, 10, or 11 βs which are

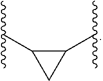 [SMILES: *C1CC1*]

7. The system of claim 6, wherein the cyclopropane compound comprises or has 3 to 10 β s which are

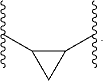 [SMILES: *C1CC1*]

8. The system of claim 7, wherein the cyclopropane compound comprises or has 5 to 7 βs which are

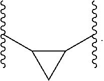 [SMILES: *C1CC1*]

9. The system of claim 1, wherein a 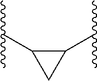 [SMILES: *C1CC1*]
is adjacent to α.

10. The system of claim 1, wherein the cyclopropane compound comprises a chain of 2, 3, 4, 5, 6, 7, 8, 9, 10, or 11 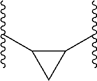 [SMILES: *C1CC1*]
s.

11. The system of claim 1, wherein the iterative PKS is an iterative PKS of Streptomyces roseoverticillatus, Streptomyces albireticali, Streptomyces sp. CC24, Streptomyces sp. V2, Streptomyces niveiscabiei NRRL B-24457, Streptomyces acidiscabies NCPPB 4445, Streptomyces sp. TLI 146, Actinobacteria bacterium OK074, Streptomyces caatingaensis CMAA 1322, Streptomyces albireticuli SMD11, Streptomyces albireticuli NRRL B-1670, Streptomyces eurocidicus ATCC 27428, Streptomyces luteoverticillatus CGMCC 15060, Streptomyces sp. 67, Streptomyces sp. 3212, Streptomyces sp. MUSC 14, Streptomyces cellostaticus DSM 40189, Streptomyces roseoverticillatus, Streptomyces klenkii KCTC 29202, Streptomyces yanglinensis CGMCC 4.2023, Streptomyces sp. CC24A, Lentzea jiangxiensis CGMCC 4.6609, or Chloroflexi bacterium HGW-Chloroflexi-6.

12. A nucleic acid comprising a biosynthetic gene cluster (BGC) encoding the iterative polyketide synthase (PKS) comprising ketosynthase (KS)-acyltransferase (AT)-dehydratase (DH)-acyl carrier protein (ACP) capable of synthesizing a cyclopropane group, a methyl transferase, a ketoreductase, and a thioesterase of claim 1.

13. A vector comprising the nucleic acid of claim 12.

14. A genetically modified host cell comprising the system of claim 1 capable of producing the cyclopropane compound.

15. The genetically modified host cell of claim 14 further comprising an O-methyltransferase, wherein the host cell produces a fatty acid methyl ester.

16. The genetically modified host cell of claim 14 further comprising a decarboxylase, wherein the host cell produces an alkene.

17. The genetically modified host cell of claim 14 further comprising a reductase, wherein the host cell produces an aldehyde.

18. The genetically modified host cell of claim 17 further comprising a decarbonylase, wherein the host cell produces an alkene.

19. A method of producing a cyclopropane compound comprising: (a) providing a genetically modified host cell of claim 14, and (b) culturing or growing the genetically modified host cell in a suitable culture or medium such that the cyclopropane compound, or a mixture thereof, is produce.

20. The method of claim 19 comprising: (c) extracting or separating the cyclopropane compound from the host cells, and/or culture or medium.

21. The method of claim 20 comprising: (d) introducing a fuel additive to the extracted or separated cyclopropane compound.


SEQUENCE LISTING (21 sequences)

SEQ ID NO: 1 - protein, 1322 aa, Streptomyces sp.
VDRHVSIVGIGCALPGGVADVEDVRGAFLHGRDCVGPIPEERWGADAFYDPDPLRPGRTYVRHGGFVDDIDAFDAAFFGISDTEAARMDPQQRLLLQTVWHALENAGQNPDELRGTSTGVFLASMNTNNYAMLKNTLQGPEGITPYDAMGDAISISAGRVAHFLGVEGPCLAVDTACSGALVALHLARQSILAGECDAAIVAGVNVMLNPGIHIAFSKVGLLSRNGQCRAFDARADGYVRSEGCVAALLRRESLAVERGDPIYATVVGTAVNHDGRTQALTAPNGRNQEQVIHRALAGVGIDTADTGYVEAHGTGTPVGDPIEMSAIVNAYGYGRPADRPLYVGSAKSNFGHIEAGAGLLGVVRAALSLQHEVIFPSIHVDQLNPRIDLRGAPVRLPSAPVPWPRGDTPRHAGVNSFGYSGTNAHAILREAPAVRPREQAAPRRATELLALSAKSAESLEELADRWAEFLAHDDIDLGAAARTAAIGRATLRHRLTVTAADSAEAAKALRRRRSGRAPVTVSEGRARRNPRVAFVFTGQGAQYPGMGRELYAAEPVFAAALDRCADVMDADLGLPLHEVLFDERVSTEALNNTQYVQPAVFAVEFALAELLKDWGVEPSVVIGHSIGELVAACTAGMLPFEEAARFAVRRGRAMGSLPPGGKMLAVAAEAAVVEAWLTGREDAVSLAAVNGPRSVVVSGAAAAVDEVARRADEAGLRTTELKVSHAFHSPLMDPALAELERAAAALHPRVPAVPVVSNVTGAPLTGTEEPAYWAAQMRDPVRFHDGMRTVVESGCSAVVEVGPHAALIPAVAAAFGQADIALVRTLLRDRQDVRNMRTAAGVLHTTGCPVRLPRLFTTGGHRRIHAPEYPFRRDRHWIAPTEGGWDLGSMMRGPRDETGPSGTWPTELAAATPWADHRVLGATVFPATGHLELALRALAAHTADAPDAPLTGTAAPATPASCEDLAFVRPLLLKPRRPTVATTALRPAPGQDGAFHFTVSTSGTGQHPVEHCRGTVRPAAAPADEPRTPPAELRAPLGAGQPPGRLYGMLREAGLEYGTSFSTVRELWPGGDGTGAALGRIRATPDGAGGAEHGHALATMLDGCLHVTAAALFTLPARLLEGAYIPVTLRRATLHRPLPEQVWSQVSVRTNDQGTAAVASARVVDDEGRLLAELDGLELRHTSALTGAADSGQAPAPARPYSGEARKLLLERLGPLGQRERVAAMGAWLLDEVRDTLGQAADDFDIDDLDPSTALLEIGLDSLMITELQRRLQEKLDFRFEAMEALTYQSLEDLAGYILDRALGPALPAAARNETEQQPEPA

SEQ ID NO: 2 - protein, 507 aa, Streptomyces sp.
MLDAGFVHTYIDTHLEQRQVNKIQHGFPSPRYWSRTDVPVEEIAEDRRRIRAAGRDSFVNFYVGVPYCIQTDPGKCGYCLFPVEEFQGNAALENYFGYVEREADLYRQALSGATLGAVYFGGGTSNLYKEPMYHRLMDLVRGLFPDIAPGADITLEGIPQLFSRAKMQAIKDSGMNRVSMGIQQVDERLNKLSGRKQTTRHVVQSLEWARELGLAANVDLIFGWPQQTVGTMLKDLETLVSWNVYDITHYELNVGGPTDFALNRFHELPSTLANLEMYRASRDFLTDQGYEQITAYNFRKPGDPAGRGYEEGVNRFLDSMDTVGLGYAAVSFFGNSAIGTDRSWSFINHLSLPRYKQALEEGRFPVERGFSHEAADWRLAMLFRSLFGLTVNRADYRAAFGTDVYEEFATVWDGLGEYGFVEVSDAEVRLVGDGPFYTPMVQALLAEERYRALRERETQAAQARRAARRARRTGNGQDGAGTEDDAAPADTGAAADAEAAAASPARG

SEQ ID NO: 3 - protein, 253 aa, Streptomyces sp.
MSEHLPLDGKRLVVTGGARGIGAGIVRLALRQGAEVVFSYNRSAERARELCAELRAAHPGQQCTALPAQVADTDSAARFAQAALEALGSVDALVNNAGVTRDGVFARMRREDWDEAVETNLGSMFTVTRPLVMALVRRRAGAIVNVTSSVGIHGAPGQANYAASKAGIIGFSKALAKELAELGVRVNAVAPGLIATDMTAGIPPERLEEIKKRIPGRQLGSVEDVAHLVCFLASDRARYITGQVIEVSGGLAH

SEQ ID NO: 4 - protein, 314 aa, Streptomyces sp.
VTTDHSTAVEDLLDVLTPDRIDARTFRPRGPVSWSSRHLYGGQVAAQALLAAGRTVAEDRPVHSLHAYFVRPGDPDAAVPLLFDVDEVRDGRAVSLRQVTARQRDAVLFTLSASFHRAEPGLDHQDLMPAVGVPEALPTYEERLARALGEPVLPLGMPFDLRYAGPLSVEAQRDPALRTGSNPLWLRTNGALPGDLPPLVHAALLTYISDILLDTVALRHGLSWADGTARPRSVDHAQWFHRPFRADDWLLLAQDTPVAYGGRALARAQVFTRGGDLVASAVQEGLVRLRRRPAAEPEGPVSGPGPRTPRSPGR

SEQ ID NO: 5 - protein, 1280 aa, Streptomyces albireticuli
VEEIPSSRWDVDELYDPDPLALGKTYVRHGGFVDDVDLFDAAFFGISDAEAARMDPQQRLLLQTVWHALEHAGQNPDEIRGSDTGVFLAMMNSNNYAFLKHDAGGLTGITAYDSMADEISISAGRIAHFLDLKGPCLTVDTACSGSLTALHLARQSILTGECDSAVVAGVNLILSPDVHVSFCKLGLFSRAGQCRAFDAKADGYVRSEGCVAALVRRESLAEERGDPILASVLGTAINHDGHTPALTAPNGRTQEQVIRTVLSRTGVDPAGVGYVEAHGTGTPVGDPIEMNAIAGAYGHARTADRPLYVGSVKSNFGHTEAAAGLLGVIKAALSLHHETIYPSLHLDRLNPKIDLKGAAVEVPGEPVPWPRGDTPRLAAVNSFGYSGTNAHAILREAPRARLGAGDTARPRPAELLVLSAKSPESLDGLADRWADYLSRADQETLPAAVFTAAGRAAHRHRLAVTGRGALGIANDLRLWRTRRTPPSVLSGHPAKPARTAFVFTGQGVQYPGMSRELHDSEPVFADAVERCAEVLDTELPVPLRRLLFEEPSPEVLDDTRLAQPALFAVEYGLATLLRSWGVVPDAVVGHSIGEVVAACVAGMLPLEDAARFSALRGRLMGELPRDGVMLAVAAPPETVRGWVSGREADVSVAAVNGPRAVVVSGRAEAVDEVARLAGAAGVRTTRLRTSHAFHSPLMDPALPELGKAAAALRPAAPVLPVLSNVTGEPLTGAEGPEYWSQQLRRPVLFHDSMRAVAALDCTVVVEIGPHPALRAHIPEAFGATGVTVIPTLSRDRKDVRNLLAAAGALFTAGAAIDLPALYRGPRHRRTSSAPLYPFRRDRYWLTDTPDAGRREPAEPAPRRRSPAPEAPAEPEPAARIVHRHEVRAGTPWVDHRILGSTVFPATAYLGLAVDAYASVNGHGSAPVELTDVGFVRPLLLAPTGTSSVQIGLEGDGPATDGRFRFAVAGGEGTPRYCQGKVGPAPRQDSAATRPEELRAAMPTELAPGRLYGLLREDGMEYGASFSTVREVWLDEAAGQALGRITAAPDGASRVGHEHGFATMLDGCLHLTAAAARDGAAKGTYIPVGVGRMVLRGALPDQVWGHVRLRTNDSGTAFTARLRVLDDTGNILAEMEDVEFRRVASLTDTSAVPAAPAGDRARESGDSRRELRERIEPLTAEERRQAVIGWLTDEIIDTLGRMSAELAVDIHHLDPSLALLEIGLDSLSITELQRRIQEKLDFRFKAMEALEYQSIEELAEYLVQRVILAEPADAATAPTDS

SEQ ID NO: 6 - protein, 484 aa, Streptomyces albireticuli
VLTPDFVNNYLDSHLAERQVNKIQHGFPSPRFWNELSVPLDEIGEDRRRLSETHNESPVFLYIGVPYCIKTDPGKCGYCLFPVEEFQGNAALENYYGYVEREAEMYREQMEGVLLAGAYFGGGTSNLYRPAVYHRIMDMVRRLFPEISDQADLTLEGIPQLFTREKMRAIADSGMNRISMGVQQINERLNSFSGRKQTTKHVIQSLEWARELGLAANVDLIFGWPQQTVDTLLEDLETLVSWDVYDITHYELNVGGPTDFALNRYHELPSTLANLELYRAGRDFLVDHGYEQLSTYNFRRPGDPTTRDFREGYTTRFDHVDSLGLGYAAITFFGNPALPSGRSWSFINHRSLPQYKAAIDNGRFPVERGFRHTPDDWLLMLLFRSLISTDIDRTRYRTALGLDIYEKFATIWDALAERGLAKVTPERIKLVDDGAFYAPMISALVAEERYRELREQAARHRRESRGHAAAAGVTLPVPGVGSGG

SEQ ID NO: 7 - protein, 253 aa, Streptomyces albireticuli
VADDLPLAGRGVIVTGGSRGIGAAVVRLALAQGADVVFGYHSGEDRARALADELRAAHPGQRCTPLYAHVADAGEAERFAGAALDRLDRFDVLVNNAGVTRDTLFARMAPQQWHEVIATNLDSMYTVTKPLLMPLVKQHSGAIVNIASSSGLHGIPGQTAYSAAKAGVIGFTKALAKEIGARGVTVNAVAPGLIETDMTAAIPEDKAEFLKSLIPGHAFGSPEDVAHLVCFLASDRARYITGQAVEVSGGLVV

SEQ ID NO: 8 - protein, 299 aa, Streptomyces albireticuli
MDRERALLDLLDLAPGGGTPGRGEGVHFVGRPPLEQSVPVYGGHLAAQALAAAGRTVPAALPAHSVHCSFLRPTLPSAPFEYRVEKVRNSASFATRRVHATQHGLEVFDLTASFHRPGPGLDHQDPMPPVPDPESLPTYEERLTTAFGEVMQPLGKPYELRFVGPLSFDTEKNPSLSSPRTRVWVRAEGELPDETAAGGARLLHACLLVYVCDVTMLETVLVRHGISWFHADGRSVDYTVWIHRPFRADDWLLCALETPAASGGRGLVLGRVFTRAGVLVATLAQEGLIRVSAGHGALG

SEQ ID NO: 9 - protein, 741 aa, Streptomyces albireticuli
MSDRFSAALYRRRTTVLWVSALALVLAALGGLGVENRLAHGGFSDPHAESSRAGRLVSEHFPTTDGDLILLLSGAGPVDSPTTASLGTDLTRRAERAAGVRAAGSYWTAGRPSALRSRDGSMGLVSLSLSGDEHAQAKTAERLVPELRRHARGLTVMAAGPAQVQTEVGKQTAHDLLLAEAIAMPITLVLLLLIFGSAVAAALPLVIALLSVLVSRAVLNALAGVVSISVYSMNSTTALGLGLGIDYSLFMLSRFREELRGGATVREALGPTVRRAGRTVAFSGLTVALSLVALLVFPQYFLRSFAYGGIVVVLSAAAGAVFVLPALLAVLGHRVDRYDVFARLRGPARRATSGTAAPVSPENGRWYRFAMAVMRRPLLYGGGAVIVLVVLASPFTRVSSGLFDDRSLPVDSQVHRATRLLRERFDRDVLRTVPVVVEGVGQSGRQALEPYARALSEVRDVRQVSAAPGAYAAGRQVRGPGAAGAALVDGDTALFSVVSAVEQDSSAGTRLVDRLRKVAPPEGSTVSVGGRAAEVRDSTSAIARATPAAIGIVVGSSLVLLFLFTGSVLMPVKALVLNTFSLSATFGAMVFVFQEGHLSPLVGSPTHTGTLDATIPILTFCVAFGLSMDYEVFLLSRIRERYLRTGDNRESVAFGLQHTGRIITAAALLVAVVLFVFAVSGVTLLKLLGVGLALAVVLDATLVRALLVPSFMRLAGRANWWAPGPLRRLHNRVGLREDADG

SEQ ID NO: 10 - protein, 402 aa, Streptomyces coelicolor
VSRRLFTSESVTEGHPDKIADQISDTILDALLREDPTSRVAVETLITTGLVHVAGEVTTKAYADIANLVRGKILEIGYDSSKKGFDGASCGVSVSIGAQSPDIAQGVDTAYENRVEGDEDELDRQGAGDQGLMFGYASDETPTLMPLPVFLAHRLSKRLSEVRKNGTIPYLRPDGKTQVTIEYDGDKAVRLDTVVVSSQHASDIDLESLLAPDIKEFVVEPELKALLEDGIKIDTENYRLLVNPTGRFEIGGPMGDAGLTGRKIIIDTYGGMARHGGGAFSGKDPSKVDRSAAYAMRWVAKNVVAAGLAARCEVQVAYAIGKAEPVGLFVETFGTAKVDTEKIEKAIDEVFDLRPAAIIRALDLLRPIYAQTAAYGHFGRELPDFTWERTDRVDALREAAGL

SEQ ID NO: 11 - protein, 541 aa, Streptomyces coelicolor
VTAPAPQPSYAHGTSTTPLLGDTVGANLGRAIAAHPDREALVDVPSGRRWTYAEFGAAVDELARGLLAKGVTRGDRVGIWAVNCPEWVLVQYATARIGVIMVNVNPAYRAHELEYVLQQSGISLLVASLAHKSSDYRAIVEQVRGRCPALRETVYIGDPSWDALTAGAAAVEQDRVDALAAELSCDDPVNIQYTSGTTGFPKGATLSHHNILNNGYWVGRTVGYTEQDRVCLPVPFYHCFGMVMGNLGATSHGACIVIPAPSSEPAATLEAVQRERCTSLYGVPTMFIAELNLPDFASYDLTSLRTGIMAGSPCPVEVMKRVVAEMHMEQVSICYGMTETSPVSLQTRMDDDLEHRTGTVGRVLPHIEVKVVDPVTGVTLPRGEAGELRTRGYSVMLGYWEEPGKTAEAIDPGRWMHTGDLAVMREDGYVEIVGRIKDMIIRGGENIYPREVEEFLYAHPKIADVQVVGVPHERYGEEVLACVVVRDAADPLTLEELRAYCAGQLAHYKVPSRLQLLDSFPMTVSGKVRKVELRERYGARP

SEQ ID NO: 12 - protein, 241 aa, Streptomyces albireticuli
MDEGGGVPGRAEASVPRILGRDPLPGGWVRGGPPQVWLLRIADHAPEPPEVYERILDADERGRATAFFRDLHRERYTAAHLGLRRLLGAYLGTGPADVALIREPCPGCGKPHGRPAVAGAPLHFNLSHAGDLAFFAFADTPVGADVEEEQPAEVVDGVVRMLHPDETAEIGALPGPDRAAAFARCWTRKEAYLKGTGTGLSESPAVTYVGSGAAPVSPPGWTLTDVAVGAGHAAAIAVATA

SEQ ID NO: 13 - protein, 241 aa, Streptomyces albireticuli; identical to SEQ ID NO: 12

SEQ ID NO: 14 - protein, 325 aa, Streptomyces coelicolor
MPAVTVENPLTLPRVSAPADAVARPVLTVTTAPSGFEGEGFPVRRAFAGINYRHLDPFIMMDQMGEVEYAPGEPKGTPWHPHRGFETVTYIVDGIFDHQDSNGGGGTITNGDTQWMTAGSGLLHIEAPPEQLVMSGGLFHGLQLWVNLPAKDKMMAPRYQDIRSGSVQLLTSPDGGALLRVIAGELDGHDGPGITHTPITMVHATLAPGAEVTLPWREDFNGLAYVMAGRGSVGAERRPVHLGQTAVFGAGGSLTVRADEKQDAHTPDLEVVLLGGRPIREPMAHYGPFVMNTKDELMQAFEDFQKGRLGTVPAVHGMSGEGPGA

SEQ ID NO: 15 - protein, 297 aa, Drosophila melanogaster
MNQASLYQHANQVQRHDAKLILDEFASTMQWRSDGEDALLDVGSGSGNVLMDFVKPLLPIRGQLVGTDISSQMVHYASKHYQREERTRFQVLDIGCERLPEELSGRFDHVTSFYCLHWVQNLKGALGNIYNLLKPEGGDCLLAFLASNPVYEVYKILKTNDKWSTFMQDVENFISPLHYSLSPGEEFSQLLNDVGFVQHNVEIRNEVFVYEGVRTLKDNVKAICPFLERMPADLHEQFLDDFIDIVISMNLQQGENNEDQKFLSPYKLVVAYARKTPEFVNNVFLEPTHQNLVKGIN

SEQ ID NO: 16 - protein, 341 aa, Synechococcus elongatus
MFGLIGHLTSLEQARDVSRRMGYDEYADQGLEFWSSAPPQIVDEITVTSATGKVIHGRYIESCFLPEMLAARRFKTATRKVLNAMSHAQKHGIDISALGGFTSIIFENFDLASLRQVRDTTLEFERFTTGNTHTAYVICRQVEAAAKTLGIDITQATVAVVGATGDIGSAVCRWLDLKLGVGDLILTARNQERLDNLQAELGRGKILPLEAALPEADFIVWVASMPQGVVIDPATLKQPCVLIDGGYPKNLGSKVQGEGIYVLNGGVVEHCFDIDWQIMSAAEMARPERQMFACFAEAMLLEFEGWHTNFSWGRNQITIEKMEAIGEASVRHGFQPLALAI

SEQ ID NO: 17 - protein, 231 aa, Synechococcus elongatus
MPQLEASLELDFQSESYKDAYSRINAIVIEGEQEAFDNYNRLAEMLPDQRDELHKLAKMEQRHMKGFMACGKNLSVTPDMGFAQKFFERLHENFKAAAAEGKVVTCLLIQSLIIECFAIAAYNIYIPVADAFARKITEGVVRDEYLHRNFGEEWLKANFDASKAELEEANRQNLPLVWLMLNEVADDARELGMERESLVEDFMIAYGEALENIGFTTREIMRMSAYGLAAV

SEQ ID NO: 18 - protein, 159 aa, Streptomyces globisporus
SNAMTATNPDYFELRHTVGFEETNLVGNVYYVNYLRWQGRCRELFLKERAPSVLAEVQEDLKLFTLKVDCEFFAEITAFDELSIRMRLSELRQTQLEFTFDYIKLGDDGGETLVARGRQRIACMRGPNTATVPTLIPEALAEALAPYSDRAGSYAGRAA

SEQ ID NO: 19 - protein, 422 aa, Jeotgalicoccus sp.
MATLKRDKGLDNTLKVLKQGYLYTTNQRNRLNTSVFQTKALGGKPFVVVTGKEGAEMFYNNDVVQREGMLPKRIVNTLFGKGAIHTVDGKKHVDRKALFMSLMTEGNLNYVRELTRTLWHANTQRMESMDEVNIYRESIVLLTKVGTRWAGVQAPPEDIERIATDMDIMIDSFRALGGAFKGYKASKEARRRVEDWLEEQIIETRKGNIHPPEGTALYEFAHWEDYLGNPMDSRTCAIDLMNTFRPLIAINRFVSFGLHAMNENPITREKIKSEPDYAYKFAQEVRRYYPFVPFLPGKAKVDIDFQGVTIPAGVGLALDVYGTTHDESLWDDPNEFRPERFETWDGSPFDLIPQGGGDYWTNHRCAGEWITVIIMEETMKYFAEKITYDVPEQDLEVDLNSIPGYVKSGFVIKNVREVVDRT

SEQ ID NO: 20 - protein, 368 aa, Mycobacterium marinum
MPREIRLPESSVVVRPAPMESATYSQSSRLQAAGLSPAITLFEKAAQTVPLPDAPQPVVIADYGVATGHNSLKPMMAAINALRRRIREDRAIMVAHTDVPDNDFTALFRTLADDPDSYLHHDSASFASAVGRSFYTQILPSNTVSLGWSSWAIQWLSRIPAGAPELTDHVQVAYSKDERARAAYAHQAATDWQDFLAFRGRELCPGGRLVVLTMALDEHGHFGYRPMNDALVAALNDQVRDGLLRPEELRRMAIPVVARAEKDLRAPFAPRGWFEGLTIEQLDVFNAEDRFWAAFQSDGDAESFGAQWAGFARAALFPTLAAALDCGTGDPRATAFIEQLEASVADRLASQPEPMRIPLASLVLAKRA

SEQ ID NO: 21 - protein, 458 aa, Acinetobacter baylyi
MRPLHPIDFIFLSLEKRQQPMHVGGLFLFQIPDNAPDTFIQDLVNDIRISKSIPVPPFNNKLNGLFWDEDEEFDLDHHFRHIALPHPGRIRELLIYISQEHSTLLDRAKPLWTCNIIEGIEGNRFAMYFKIHHAMVDGVAGMRLIEKSLSHDVTEKSIVPPWCVEGKRAKRLREPKTGKIKKIMSGIKSQLQATPTVIQELSQTVFKDIGRNPDHVSSFQAPCSILNQRVSSSRRFAAQSFDLDRFRNIAKSLNVTINDVVLAVCSGALRAYLMSHNSLPSKPLIAMVPASIRNDDSDVSNRITMILANLATHKDDPLQRLEIIRRSVQNSKQRFKRMTSDQILNYSAVVYGPAGLNIISGMMPKRQAFNLVISNVPGPREPLYWNGAKLDALYPASIVLDGQALNITMTSYLDKLEVGLIACRNALPRMQNLLTHLEEEIQLFEGVIAKQEDIKTAN